(19) United States
(12) Patent Application Publication
(10) Pub. No.: US 2025/0283078 A1
(43) Pub. Date: Sep. 11, 2025

(54) RNA COMPOSITIONS AND THERAPEUTIC METHODS THEREOF

(71) Applicant: Board of Regents of the University of Nebraska, Lincoln, NE (US)

(72) Inventors: So-Youn Kim, Omaha, NE (US); Seok-Yeong Yu, Omaha, NE (US)

(21) Appl. No.: 18/862,778

(22) PCT Filed: May 25, 2023
(86) PCT No.: PCT/US23/67481

§ 371 Date: Nov. 4, 2024

Related U.S. Application Data
(60) Provisional application No. 63/345,978, filed on May 26, 2022.

(51) Int. Cl. C12N 15/113; A61P 35/00

(52) U.S. Cl. CPC C12N 15/113; A61P 35/00; C12N 2310/14; C12N 2310/51

(57) ABSTRACT

Inhibitory nucleic acid molecules are provided as well as methods of use thereof. Specifically, the disclose provides an Inhibin BetaA (inhba) inhibitory nucleic acid molecule, which is an antisense oligonucleotide, siRNA, or shRNA. Further disclosed are sequences that are targeted by the inhba inhibitory nucleic acid molecule; and methods of treating a disease or disorder in a subject comprising administering an inhba inhibitory nucleic acid molecule to the subject, wherein the disease or disorder is selected from the group consisting of cancers, autoimmune diseases, fibrotic disorders, blood disorders, allergies and allergic diseases, heart failure, neurodegenerative diseases, cachexia and inflammatory diseases.

This application claims priority under 35 U.S.C. § 119(e) to U.S. Provisional Patent Application No. 63/345,978, filed May 26, 2022. The foregoing application is incorporated by reference herein.

This invention was made with government support under Grant No. P30 GM127200 awarded by the National Institutes of Health. The government has certain rights in the invention.

### FIELD OF THE INVENTION

This application relates to the field of therapeutics. More specifically, this invention provides inhibitory nucleic acid molecules and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Activin A is comprised of a dimer of the Inhibin βA (INHBA) subunit and is a cytokine of the TGFβ superfamily. Activin A is expressed in numerous tissues and plays pivotal roles in regulation of tissue homeostasis, organ development, inflammation, and cell proliferation (Bloise, et al. (2019) Physiol. Rev., 99:739-780). The activin signaling pathway has been researched extensively regarding its functions in pancreatic cancer development and progression (Qiu, et al. (2021) Biomedicines 9:821). Activin signaling can be exploited by cancer cells for their growth advantage paradoxically, despite its evident role in tumor suppression (Mancinelli, et al. (2021) Sci. Rep., 11:7986; Zhao, et al. (2020) Can. Res., 80:3359-3371). Multiple studies have implicated a significant role of activin A in skeletal muscle wasting in cancer-associated cachexia and led to propose serum levels of activin A as a prognostic biomarker for cancer patients (Loumaye, et al. (2017) J. Cachexia. Sarcopenia Muscle 8:768-777; Chen, et al. (2014) FASEB J., 28:1711-1723; Narasimhan, et al. (2020) Cancers 12:3787).

### SUMMARY OF THE INVENTION

In accordance with the instant invention, inhibitors of inhibin RA (inhba) are provided. In certain embodiments, the inhibitors are inhibitory nucleic acid molecule. In certain embodiments, the inhibitory nucleic acid molecule is an antisense oligonucleotide, siRNA, or shRNA. In certain embodiments, the inhibitory nucleic acid molecule targets a sequence in SEQ ID NO: 2 or SEQ ID NO: 3. In certain embodiments, the inhibitory nucleic acid molecule comprises a sequence which is the complement of a sequence in SEQ ID NO: 2 or SEQ ID NO: 3. In certain embodiments, the inhibitory nucleic acid molecule targets SEQ ID NO: 4, SEQ ID NO: 6, SEQ ID NO: 8, or SEQ ID NO: 10. In certain embodiments, the inhibitory nucleic acid molecule comprises a modified linkage, modified sugar group, a modified base, and/or modified phosphate group. Vehicles, such as nanoparticles, micelles, microparticles, lipid polymers, or hydrogels, comprising an inhba inhibitory nucleic acid molecule are also encompassed by the instant invention. Compositions comprising an inhba inhibitory nucleic acid molecule and at least one carrier (e.g., a pharmaceutically acceptable carrier) are also provided.

In accordance with another aspect of the instant invention, methods of treating a disease or disorder in a subject are provided. In certain embodiments, the method comprises administering a therapeutically effective amount of an inhba inhibitory nucleic acid molecule to the subject. In certain embodiments, the disease or disorder is selected from the group consisting of cancers, autoimmune diseases, fibrotic disorders, blood disorders, allergies and allergic diseases, heart failure, neurodegenerative diseases, cachexia and inflammatory diseases. In certain embodiments, the disease or disorder is cancer, particularly cancers of the pancreas, colon, liver, and the head and neck. In certain embodiments, the disease or disorder is pancreatic ductal adenocarcinoma. In certain embodiments, the disease or disorder is cachexia.

### BRIEF DESCRIPTION OF THE DRAWINGS

FIG. 1A provides the gene expression for inhibin subunit isoforms and FST. Outliers were removed using GraphPad (α=0.05). FIG. 1B provides Kaplan-Meier overall survival plots by INHBA mRNA expression. FIG. 1C provides graphs of the correlation between INHBA and KRAS or TP53 genes. ***P<0.001; n.s., not significant.

FIGS. 2A and 2B provide the medium Activin A, IL-6, and GDF15 levels from different KPC cell lines (n=3/line). FIGS. 2C-2E provides an RT-PCR analysis of KPC cell lines (n=3/line) with the reference of normal pancreas. FIG. 2F provides an RT-PCR analysis of KPC cell lines (n=3/line) with the reference of normal pancreas. FIG. 2G provides a graph of the stained area of each membrane calculated by ImageJ and expressed as arbitrary units. *P<0.05; **P<0.01; ***P<0.001; ****P<0.0001; n.s., not significant.

FIG. 3A shows the screening of inhibitory effects of four different inhba siRNAs in KPC8069 cells. FIG. 3B shows inhba expression in KPC8069 cells treated with either scramble (Sc-si) or inhba siRNA (Inhba-si) at 10 nM for 48 hours. The gene expression of inhba was normalized with 18s. FIG. 3C shows Activin A levels in media measured using ELISA kit after 48-hour treatment of designated siRNA.

FIG. 4A provides a schematic of the experimental design. 12-week-old male mice were subject to surgery. All the mice received the active procedure for KPC8069 cell implantation into the pancreas except for the sham group which underwent the surgical procedures without cell implantation. The mice were randomly divided into groups and were intraperitoneally treated with designated treatments. FIGS. 4B and 4C provide graphs of pancreas weight uncorrected and corrected, respectively, for body weight (%). FIG. 4D provides representative gross appearance of tumors from orthotopic mice. Lines and arrows indicate tumors. FIGS. 4E-4H provide graphs of ferum activin A, inhibin A, the ratio of activin A to inhibin A, and tissue activin A levels. FIGS. 4I and 4J provide graphs of serum GDF15 and IL-6 levels. FIG. 4K provides a graph of the average daily body weights: P<0.01 for Sham and Sc-si groups (A, C) and P<0.01 for Sc-si and Inhba-si groups (C, D). FIG. 4L provides a graph of body weight changes. FIG. 4M provides a graph of the average number of metastasized tissues (per mouse). FIG. 4N provides a graph of Chi-square test for the total number of metastases between Sc-si and Inhba-si groups. FIG. 4O provides a graph of the average number of metastasized tissues and histologically confirmed invasive metastasis. FIG. 4P provides a survival test scheme. Orthotopic mice were randomly divided into two groups, were intraperitoneally treated with either scramble siRNA or inhba siRNA every other day from D11 to D17, and were followed to record survival probabilities for groups. FIG. 4Q provides a Kaplan-Meier survival curve for the survival of Sc-si and Inhba-si groups with the Mantel-Cox test. FIG. 4R provides a graph of the size distribution of adipocytes (top) and the size distribution of muscle fiber (bottom). FIGS. 4S and 4T provide graphs of the body weight change and food intake, respectively, during survival test. Inhba-si extends to 22 days while Sc-si terminated at 20 days. *P<0.05; **P<0.01; ***P<0.001; n.s., not significant.

FIG. 5A provides fluorescence intensities of indicated tissue from orthotopic mice intraperitoneally injected with Cy5.5-labeled scramble siRNA at D10, as measured by ImageJ. FIG. 5B provides graphs of relative protein expression that is normalized by ponceau S staining from Sc-si and Inhba-si groups. *P<0.05; n.s., not significant.

FIG. 6A provides graphs of the number of Ki67-positive cells among CK19-positive, negative, or both cells from the field of view, FOV (n=5/group). FIG. 6B provides a graph of the absorbance of KPC8069 (left) and KPC1245 (right). FIG. 6C provides a graph of the absorbance of KPC8069 (top) and KPC1245 (bottom) with exogenous activin A and/or SIS3. *P<0.05; **P<0.01; ***P<0.001; n.s., not significant.

FIG. 7A provides a graph of the mean fluorescence intensity of α-SMA in tumor sections. Field of view (FOV; n=3/group). FIG. 7B provides a graph of the fluorescence intensity of BAX in tumor sections. **P<0.01; ***P<0.001.

FIG. 8A provides a graph of PSC proliferation by inhba siRNA. FIG. 8B provides a Western blot analysis of PSCs stimulated with activin A (5 ng mL⁻¹) in the absence or presence of SIS3 (0.5 μm). FIGS. 8C and 8D provides graphs of relative protein expression after normalization with ponceau S staining. FIG. 8E provides a graph of the absorbance of crystal violet from KPC 8069 24 hours after the treatment with conditioned media from PSCs. In vitro data were obtained from three independent experiments (n=3). *P<0.05; **P<0.01; n.s., not significant.

FIG. 9 shows that Activin A upregulates SOX9 and decreases ACVR2B in 266-6 acinar cells. Western blot analysis of pSMAD3, SMAD3, SOX9 and ACVR2B in 266-6 acinar cells treated with Activin A (5 ng/mL) for 1 hour are provided (three independent experiments, n=3). * P<0.05.

### DETAILED DESCRIPTION OF THE INVENTION

The present invention provides inhibitors of Inhibin βA (inhba). The inhibition of inhba also results in the inhibition of activin A, which comprises a dimer of inhba. Inhba inhibitors are compounds which reduce inhba activity and/or the expression of inhba. In certain embodiments, the inhba inhibitors are inhibitory nucleic acid molecules. Examples of inhibitory nucleic acid molecule include, without limitation, antisense oligonucleotides, microRNA, and RNAi constructs such as siRNA or shRNA molecules. The present invention also encompasses nucleic acid molecules comprising the inhibitory nucleic acid molecule and nucleic acid molecules encoding the inhibitory nucleic acid molecule.

The inhibition of the expression of the inhba gene transcript and/or inhba protein refers to the reduction of the expression of the inhba gene transcript and/or inhba protein (e.g., in a cell or a tissue). In certain embodiments, the term “inhibiting” refers to at least 5%, at least 10%, at least 15%, at least 20%, at least 25%, at least 30%, at least 35%, at least 40%, at least 45%, at least 50%, at least 60%, at least 70%, at least 80%, at least 90%, at least 95%, at least 99%, or 100% (complete) inhibition of inhba gene transcript and/or inhba protein expression (e.g., in a cell or a tissue).

Examples of nucleotide and amino acid sequences for human Inhibin βA (inhba) are provided in GenBank Gene ID: 3624, including all variants and isoforms described therein. In certain embodiments, the inhba nucleotide and/or amino acid sequence is mammalian. In certain embodiments, the inhba nucleotide and/or amino acid sequence is human. In certain embodiments, the inhba nucleotide and/or amino acid sequence is mouse, rat, rabbit, or porcine. In certain embodiments, the inhba nucleotide and amino acid sequences are provided in GenBank Accession No. NM_002192.4 and/or GenBank Accession No. NP_002183.1. In certain embodiments, the amino acid sequence of inhba is:

| (SEQ ID NO: 1) | |
|---|---|
| 1 | MPLLWLRGFL LASCWIIVRS SPTPGSEGHS AAPDCPSCAL AALPKDVPNS QPEMVEAVKK |
| 61 | HILNMLHLKK RPDVTQPVPK AALLNAIRKL HVGKVGENGY VEIEDDIGRR AEMNELMEQT |
| 121 | SEIITFAESG TARKTLHFEI SKEGSDLSVV ERAEVWLFLK VPKANRTRTK VTIRLFQQQK |
| 181 | HPQGSLDTGE EAEEVGLKGE RSELLLSEKV VDARKSTWHV FPVSSSIQRL LDQGKSSLDV |
| 241 | RIACEQCQES GASLVLLGKK KKKEEEGEGK KKGGGEGGAG ADEEKEQSHR PFLMLQARQS |
| 301 | EDHPHRRRRR GLECDGKVNI CCKKQFFVSF KDIGWNDWII APSGYHANYC EGECPSHIAG |
| 361 | TSGSSLSFHS TVINHYRMRG HSPFANLKSC CVPTKLRPMS MLYYDDGQNI IKKDIQNMIV |
| 421 | EECGCS |

Notably, amino acids 1-20 are a signal peptide.

In certain embodiments, the nucleotide sequence of inhba is:

| (SEQ ID NO: 2) | |
|---|---|
| 1 | acagtgccaa taccatgaag aggagctcag acagctctta ccacatgata caagagccgg |
| 61 | ctggtggaag agtggggacc agaaagagaa tttgctgaag aggagaagga aaaaaaaaac |
| 121 | accaaaaaaa aaaataaaaa aatccacaca cacaaaaaaa cctgcgcgtg aggggggagg |
| 181 | aaaagcaggg ccttttaaaa aggcaatcac aacaactttt gctgccagga tgcccttgct |
| 241 | ttggctgaga ggatttctgt tggcaagttg ctggattata gtgaggagtt cccccacccc |
| 301 | aggatccgag gggcacagcg cggcccccga ctgtccgtcc tgtgcgctgg ccgccctccc |
| 361 | aaaggatgta cccaactctc agccagagat ggtggaggcc gtcaagaagc acattttaaa |
| 421 | catgctgcac ttgaagaaga gacccgatgt cacccagccg gtacccaagg cggcgcttct |
| 481 | gaacgcgatc agaaagcttc atgtgggcaa agtcggggag aacgggtatg tggagataga |
| 541 | ggatgacatt ggaaggaggg cagaaatgaa tgaacttatg gagcagacct cggagatcat |
| 601 | cacgtttgcc gagtcaggaa cagccaggaa gacgctgcac ttcgagattt ccaaggaagg |
| 661 | cagtgacctg tcagtggtgg agcgtgcaga agtctggctc ttcctaaaag tccccaaggc |
| 721 | caacaggacc aggaccaaag tcaccatccg cctcttccag cagcagaagc acccgcaggg |
| 781 | cagcttggac acaggggaag aggccgagga agtgggctta aagggggaga ggagtgaact |
| 841 | gttgctctct gaaaaagtag tagacgctcg gaagagcacc tggcatgtct tccctgtctc |
| 901 | cagcagcatc cagcggttgc tggaccaggg caagagctcc ctggacgttc ggattgcctg |
| 961 | tgagcagtgc caggagagtg gcgccagctt ggttctcctg ggcaagaaga agaagaaaga |
| 1021 | agaggagggg gaagggaaaa agaagggcgg aggtgaaggt ggggcaggag cagatgagga |
| 1081 | aaaggagcag tcgcacagac ctttcctcat gctgcaggcc cggcagtctg aagaccaccc |
| 1141 | tcatcgccgg cgtcggcggg gcttggagtg tgatggcaag gtcaacatct gctgtaagaa |
| 1201 | acagttcttt gtcagtttca aggacatcgg ctggaatgac tggatcattg ctccctctgg |
| 1261 | ctatcatgcc aactactgcg agggtgagtg cccgagccat atagcaggca cgtccgggtc |
| 1321 | ctcactgtcc ttccactcaa cagtcatcaa ccactaccgc atgcggggcc atagcccctt |
| 1381 | tgccaacctc aaatcgtgct gtgtgcccac caagctgaga cccatgtcca tgttgtacta |
| 1441 | tgatgatggt caaaacatca tcaaaaagga cattcagaac atgatcgtgg aggagtgtgg |
| 1501 | gtgctcatag agttgcccag cccaggggga aagggagcaa gagttgtcca gagaagacag |
| 1561 | tggcaaaatg aagaaatttt taaggtttct gagttaacca gaaaaataga aattaaaaac |
| 1621 | aaaacaaaaa aaaaaacaaa aaaaaacaaa agtaaattaa aaacaaaacc tgatgaaaca |
| 1681 | gatgaaggaa gatgtggaaa aaatccttag ccagggctca gagatgaagc agtgaaagag |
| 1741 | acaggaattg ggagggaaag ggagaatggt gtacccttta tttcttctga aatcacactg |
| 1801 | atgacatcag ttgtttaaac ggggtattgt cctttccccc cttgaggttc ccttgtgagc |
| 1861 | cttgaatcaa ccaatctagt ctgcagtagt gtggactaga acaacccaaa tagcatctag |
| 1921 | aaagccatga gtttgaaagg gcccatcaca ggcactttcc tacccaatta cccaggtcat |
| 1981 | aaggtatgtc tgtgtgacac ttatctctgt gtatatcagc atacacacac acacacacac |
| 2041 | acacacacac acacacaggc atttccacac attacatata tacacatact ggtaaaagaa |
| 2101 | caatcgtgtg caggtggtca cacttccttt ttctgtacca cttttgcaac aaaacaaaac |
| 2161 | aaacaacatt aaaaaattga gaacaagtat ggaaagaatg aaagatcaag gaaaaaagaa |
| 2221 | taccaagtta catttcgtta aggtgcttat gatcttagaa ctatgcaacc taataggttt |
| 2281 | gaaactgttt acctgagaga gaacaaaaag agagactttt ttgtattgga agtaatctga |
| 2341 | ttaattttta ttttcttcaa ggagagatac ttgaaaggaa tatgtttgtc catctgttgg |
| 2401 | atccaaacat ttctatattt tgtaaatgtt gttgttgttt tttttttaat cgtttactat |
| 2461 | ttgcactaca atggtgtttg acctgtctaa tccttattta acaagtattt ttttggttg |
| 2521 | ggggtggggg tggggtttaa gagctgcact taatgtgagc tataaaagaa ctgctacagc |
| 2581 | acacaaaata gctattttta ttattataat tataattatt attattattt tgtaccttaa |
| 2641 | aaaatagaca catacaccaa agacatttgt gtgagccttt aaacagtctg tctgtggttg |
| 2701 | gtatcattca ccatcaatga gtcaggggtt gggattcaag gttgagtagt gtggattgtg |
| 2761 | ttcaggctta aaagacctga gaagtttggt ttttgactcc ttttacatcc atgaaacagg |
| 2821 | acatttcata ctggatgtac agtagttgta cactgttgga tatcaagttc aatcaaattc |
| 2881 | atggaactac atgcttgtat gtgtatatat acattgcttg tgcatatgca tatctgtatg |
| 2941 | tatatataca tgtattgtac catgtccata cacattttaa gcacttcagg ctgtcatttt |
| 3001 | ttaatgttct taaagcaatg aatgtttgtg tgcaaaacac agtattttta agaaggatag |
| 3061 | gctatagttt ttgcttttac tctgaactag gtgggcgcat ttcaaaaatt cggatgggaa |
| 3121 | aaagcctgga aattccagtg aatattcagc aaggccctct ttcattgtac agggatcaaa |
| 3181 | tttcctcctc ttttttgtgc cccctcccac ttctacaagt tatcccctgt ggggaaaaca |
| 3241 | ggatgataat caaaactctg ggctgatgtt tttccaactt agtgtctatt ggaatcaatc |
| 3301 | ttaaatcaga agctttttca gaaaaataat atttaggcca gaattagagt tgagtgtatt |
| 3361 | ttttaaaaat gattaaggct tggttgtgag aaatattacc tgtaccagct gggaaaaata |
| 3421 | atgtcatcac taactaaaag ataattaatt tgagagaaag tgttaagaga gggagagtaa |
| 3481 | ggaagagaac agttaagagg aggcagaggt gagggcagta gtaaaaatct ctaaaatttt |
| 3541 | aatttacagc caaaattctt catgtgtaaa tttgtattga ttcagatgca gaaatgaaaa |
| 3601 | aaaaacacct ttgttttata aatatcaaag tacatgctta aagccaagtt tttatctagt |
| 3661 | ttattctagt acttagcttg cctggaatag ctaataaatt attcatgtat gtgcttttga |
| 3721 | aaatccagag ccctattttt acacacttgt gtgaagttgg caaacatttt gaaaaatgga |
| 3781 | aaaaagtttc taataattgg gaacaattac attaattaat attttgtaaa atattgaagc |
| 3841 | ttttagccct atgtcaattt gtagattaaa ataaattaat tataggaaag gaagataaca |
| 3901 | gtgagaaacc aaacattaca aaaggtggtt tagctctcct tgaaaaatat actaagttgg |
| 3961 | tatactataa cacttggcta tatgtaggca atgtcactac tgggcaaata cacttactgt |
| 4021 | gttctagagg cagccctttc ttatgcagaa aatacaatac gcactgcatg agaagcttga |
| 4081 | gagtggattc taatccaggt ctgtcgacct tggatatcat gcatgtggga aggtgggtgt |
| 4141 | ggtgagaaaa gttttaaggc aagagtagat ggccatgttc aactttacaa aatttcttgg |
| 4201 | aaaactggca gtattttgaa ctgcatcttc tttggtaccg gaacctgcag aaacagtgtg |
| 4261 | agaaattaag tcctggttca ctgcgcagta gcaaagatgg tcaaggccat ggaaaaagca |
| 4321 | gaaatttacc aagaaagctg atacccatgt atagttccca ctcatctcaa atacatctgc |
| 4381 | tatcttttta agctaagtcc tagacatatc ggggataaca tgggggttga ttagtgacca |
| 4441 | cagttatcag aagcagagaa atgtaattcc atattttatt tgaaacttat tccatatttt |
| 4501 | aattggatat tgagtgattg ggttatcaaa cacccacaaa ctttaatttt gttaaattta |
| 4561 | tatggctttg aaatagaagt ataagttgct accatttttt gataacattg aaagatagta |
| 4621 | ttttaccatc tttaatcatc ttggaaaata caagtcctgt gaacaaccac tctttcacct |
| 4681 | agcagcatga ggccaaaagt aaaggcttta aattataaca tatgggattc ttagtagtat |
| 4741 | gtttttttct tgaaactcag tggctctatc taaccttact atctcctcac tctttctcta |
| 4801 | agactaaact ctaggctctt aaaaatctgc ccacaccaat cttagaagct ctgaaaagaa |
| 4861 | tttgtcttta aatatctttt aatagtaaca tgtattttat ggaccaaatt gacattttcg |
| 4921 | actatttttt ccaaaaaagt caggtgaatt tcagcacact gagttgggaa tttcttatcc |
| 4981 | cagaagacca accaatttca tatttattta agattgattc catactccgt tttcaaggag |
| 5041 | aatccctgca gtctccttaa aggtagaaca aatactttct attttttttt caccattgtg |
| 5101 | ggattggact ttaagaggtg actctaaaaa aacagagaac aaatatgtct cagttgtatt |
| 5161 | aagcacggac ccatattatc atattcactt aaaaaaatga tttcctgtgc accttttggc |
| 5221 | aacttctctt ttcaatgtag ggaaaaactt agtcaccctg aaaacccaca aaataaataa |
| 5281 | aacttgtaga tgtgggcaga aggtttgggg gtggacattg tatgtgttta aattaaaccc |
| 5341 | tgtatcactg agaagctgtt gtatgggtca gagaaaatga atgcttagaa gctgttcaca |
| 5401 | tcttcaagag cagaagcaaa ccacatgtct cagctatatt attatttatt ttttatgcat |
| 5461 | aaagtgaatc atttcttctg tattaatttc caaagggttt taccctctat ttaaatgctt |
| 5521 | tgaaaaacag tgcattgaca atgggttgat atttttcttt aaaagaaaaa tataattatg |
| 5581 | aaagccaaga taatctgaag cctgttttat tttaaaactt tttatgttct gtggttgatg |
| 5641 | ttgtttgttt gtttgtttct attttgttgg ttttttactt tgttttttgt tttgttttgt |
| 5701 | tttgttttgc atactacatg cagttcttta accaatgtct gtttggctaa tgtaattaaa |
| 5761 | gttgttaatt tatatgagtg catttcaact atgtcaatgg tttcttaata tttattgtgt |
| 5821 | agaagtactg gtaatttttt tatttacaat atgtttaaag agataacagt ttgatatgtt |
| 5881 | ttcatgtgtt tatagcagaa gttatttatt tctatggcat tccagcggat attttggtgt |
| 5941 | ttgcgaggca tgcagtcaat attttgtaca gttagtggac agtattcagc aacgcctgat |
| 6001 | agcttctttg gccttatgtt aaataaaaag acctgtttgg gatgta |

In certain embodiments, the nucleotide sequence of inhba is:

(SEQ ID NO: 3)
atgcccttgct ttggctgaga ggatttctgt tggcaagttg ctggattata gtgaggagtt
cccccacccc aggatccgag gggcacagcg cggcccccga ctgtccgtcc tgtgcgctgg
ccgccctccc aaaggatgta cccaactctc agccagagat ggtggaggcc gtcaagaagc
acattttaaa catgctgcac ttgaagaaga gacccgatgt cacccagccg gtacccaagg
cggcgcttct gaacgcgatc agaaagcttc atgtgggcaa agtcggggag aacgggtatg
tggagataga ggatgacatt ggaaggaggg cagaaatgaa tgaacttatg gagcagacct
cggagatcat cacgtttgcc gagtcaggaa cagccaggaa gacgctgcac ttcgagattt
ccaaggaagg cagtgacctg tcagtggtgg agcgtgcaga agtctggctc ttcctaaaag
tccccaaggc caacaggacc aggaccaaag tcaccatccg cctcttccag cagcagaagc
acccgcaggg cagcttggac acaggggaag aggccgagga agtgggctta aagggggaga
ggagtgaact gttgctctct gaaaaagtag tagacgctcg gaagagcacc tggcatgtct
tccctgtctc cagcagcatc cagcggttgc tggaccaggg caagagctcc ctggacgttc
ggattgcctg tgagcagtgc caggagagtg gcgccagctt ggttctcctg ggcaagaaga
agaagaaaga agaggagggg gaagggaaaa agaagggcgg aggtgaaggt ggggcaggag
cagatgagga aaaggagcag tcgcacagac ctttcctcat gctgcaggcc cggcagtctg
aagaccaccc tcatcgccgg cgtcggcggg gcttggagtg tgatggcaag gtcaacatct
gctgtaagaa acagttcttt gtcagtttca aggacatcgg ctggaatgac tggatcattg
ctccctctgg ctatcatgcc aactactgcg agggtgagtg cccgagccat atagcaggca
cgtccgggtc ctcactgtcc ttccactcaa cagtcatcaa ccactaccgc atgcggggcc
atagcccctt tgccaacctc aaatcgtgct gtgtgcccac caagctgaga cccatgtcca
tgttgtacta tgatgatggt caaaacatca tcaaaaagga cattcagaac atgatcgtgg
aggagtgtgg gtgctcatag

In certain embodiments, the inhba has at least 90%, 95%, 97%, 99%, or 100% identity, particularly at least 97% or 99% identity, with SEQ ID NO: 1, SEQ ID NO: 2, or SEQ ID NO: 3.

As stated hereinabove, examples of inhibitory nucleic acid molecules include, without limitation, antisense oligonucleotides, microRNA, siRNA, and shRNA. In certain embodiments, the inhibitory nucleic acid molecule targets a sequence within SEQ ID NO: 2 or 3. In certain embodiments, the inhibitory nucleic acid molecule targets SEQ ID NO: 4, 6, 8, or 10. In certain embodiments, the inhibitory nucleic acid molecule comprises a sequence which is the complement (complete complement) of a sequence within SEQ ID NO: 2 or 3. In certain embodiments, the inhibitory nucleic acid molecule comprises a sequence (consecutive nucleotides) within SEQ ID NO: 2 or 3. In certain embodiments, the inhibitory nucleic acid molecule comprises about 10 to about 50 nucleotides.

Antisense nucleic acid molecules, also referred to as antisense oligonucleotides, are well known in the art. Antisense oligonucleotides are single-stranded oligonucleotides which target or are complementary to a target nucleic acid molecule such as an mRNA. The antisense oligonucleotide may comprise DNA and/or RNA. In certain embodiments, the antisense oligonucleotide comprises DNA. Antisense oligonucleotides targeted to any known nucleotide sequence can be prepared by methods known in the art including oligonucleotide synthesis according to standard methods.

Antisense oligonucleotides can be targeted to any portion of an RNA or mRNA. In certain embodiments, the antisense oligonucleotide targets a translation initiation site and/or splice site. Antisense oligonucleotides are typically between about 10 and about 50 nucleotides in length. In certain embodiments, the antisense oligonucleotide is about 10 to about 45, about 10 to about 40, about 10 or about 35, about 10 to about 30, about 15 to about 30, about 15 to about 25, about 15 to about 22, about 15 to about 20, about 10 to about 25, about 12 to about 25, about 14 to about 25, or about 15 to about 25 nucleotides in length. In certain embodiments, the antisense oligonucleotide is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In certain embodiments, the antisense oligonucleotide targets (is complementary to) a sequence within an inhba nucleic acid molecule or within SEQ ID NO: 2 or 3. In certain embodiments, the antisense oligonucleotide comprises a sequence within an inhba nucleic acid molecule or within SEQ ID NO: 2 or 3.

Small, interfering RNA (siRNA) are well known in the art. siRNA are double stranded RNA molecules and generally operate within the RNA interference (RNAi) pathway and cause the degradation of a targeted nucleic acid molecule (e.g., mRNA after transcription, thereby preventing translation of a gene transcript to protein). Methods of identifying and synthesizing siRNA molecules are known in the art (see, e.g., Ausubel et al., Current Protocols in Molecular Biology, John Wiley and Sons, Inc). In certain embodiments, one or both of the strands of the siRNA may have a short overhang (e.g., 1, 2, or 3 nucleotides, typically 2) at the 5′ and/or 3′ end of the nucleotide sequence.

siRNA are typically less than about 30 nucleotides in length. In certain embodiments, the siRNA are about 12 to about 30, about 15 to about 30, about 20 to about 30, about 15 to about 25, or about 20 to about 25 nucleotides in length. In certain embodiments, the siRNA is 10, 11, 12, 13, 14, 15, 16, 17, 18, 19, 20, 21, 22, 23, 24, 25, 26, 27, 28, 29, or 30 nucleotides in length. In certain embodiments, the siRNA targets (is complementary to) a sequence within an inhba nucleic acid molecule or within SEQ ID NO: 2 or 3. In certain embodiments, the siRNA comprises a sequence within an inhba nucleic acid molecule or within SEQ ID NO: 2 or 3.

Short hairpin RNA molecules (shRNA) are single-stranded oligonucleotides which comprise each strand of an siRNA separated by a small loop or hairpin sequence (e.g., 6-15 nucleotides, particularly 7-10 nucleotides). shRNA molecules are typically processed into an siRNA within the cell by endonucleases.

In certain embodiments, the inhibitory nucleic acid molecule of the instant invention comprises a sequence selected from (or the RNA equivalent (e.g., replacing thymine (T) with uracil (U)) (e.g., SEQ ID NOs: 20-27)):

(SEQ ID NO: 4)
5′-GTCAAGAAGCACATTTTAAACATGC-3′,
(SEQ ID NO: 5)
3′-GGCAGTTCTTCGTGTAAAATTTGTACG-5′,
(SEQ ID NO: 6)
5′-AACGCGATCAGAAAGCTTCATGTGG-3′,
(SEQ ID NO: 7)
3′-ACTTGCGCTAGTCTTTCGAAGTACACC-5′,
(SEQ ID NO: 8)
5′-CTAAAAGTCCCCAAGGCCAACAGGA-3′,
(SEQ ID NO: 9)
3′-AGGATTTTCAGGGGTTCCGGTTGTCCT-5′,
(SEQ ID NO: 10)
5′-ATGATGATGGTCAAAACATCATCAA-3′,
or
(SEQ ID NO: 11)
3′-GATACTACTACCAGTTTTGTAGTAGTT-5′.

In certain embodiments, the inhibitory nucleic acid molecule comprises a sequence with at least 80%, 85%, 90%, or 95%, particularly at least 90%, 95%, 97%, or 99%, more particularly at least 97% or 99% identity with SEQ ID NO: 4, 5, 6, 7, 8, 9, 10, or 11 (or the RNA equivalent). In certain embodiments, the inhibitory nucleic acid molecule comprises a sequence which is a portion of SEQ ID NO: 4, 5, 6, 7, 8, 9, 10, or 11 (or the RNA equivalent). For example, the inhibitory nucleic acid molecule may comprise a sequence which is SEQ ID NO: 4, 5, 6, 7, 8, 9, 10, or 11 (or the RNA equivalent) wherein the terminal 1, 2, 3, 4, 5, 6, 7, 8, 9, or 10 nucleotides are deleted/absent from the 5′ and/or 3′ end of SEQ ID NO: 4, 5, 6, 7, 8, 9, 10, or 11 (or the RNA equivalent). In certain embodiments, the inhibitory nucleic acid molecule comprises SEQ ID NO: 4, 5, 6, 7, 8, 9, 10, or 11 (or the RNA equivalent) plus the nucleotides (e.g., 1, 2, 3, 4, 5, 6, 7, 8, 9, or 10 nucleotides) at the 5′ and/or 3′ present in SEQ ID NO: 2 or 3 (or the RNA equivalent).

In certain embodiments, the inhibitory nucleic acid molecule is an siRNA or shRNA which comprises:

(SEQ ID NO: 20)
5′-GUCAAGAAGCACAUUUUAAACAUGC-3′
and
(SEQ ID NO: 21)
3′-GGCAGUUCUUCGUGUAAAAUUUGUACG-5′.

In certain embodiments, the inhibitory nucleic acid molecule is an siRNA or shRNA which comprises:

(SEQ ID NO: 22)
5′-AACGCGAUCAGAAAGCUUCAUGUGG-3′,
and
(SEQ ID NO: 23)
3′-ACUUGCGCUAGUCUUUCGAAGUACACC-5′.

In certain embodiments, the inhibitory nucleic acid molecule is an siRNA or shRNA which comprises:

(SEQ ID NO: 24)
5′-CUAAAAGUCCCCAAGGCCAACAGGA-3′,
and
(SEQ ID NO: 25)
3′-AGGAUUUUCAGGGGUUCCGGUUGUCCU-5′.

In certain embodiments, the inhibitory nucleic acid molecule is an siRNA or shRNA which comprises:

(SEQ ID NO: 26)
5′-AUGAUGAUGGUCAAAACAUCAUCAA-3′,
and
(SEQ ID NO: 27)
3′ -GAUACUACUACCAGUUUUGUAGUAGUU-5′.

In certain embodiments, the inhibitory nucleic acid molecule of the instant invention comprises a sequence selected from (or the RNA equivalent (e.g., replacing thymine (T) with uracil (U)) (e.g., SEQ ID NOs: 28-35)):

(SEQ ID NO: 12)
5′-GTCAAGAAGCACATCTTAAACATGC-3′,
(SEQ ID NO: 13)
3′-GACAGTTCTTCGTGTAGAATTTGTACG-5′,
(SEQ ID NO: 14)
5′-AACGCGATCAGAAAGCTTCATGTGG-3′,
(SEQ ID NO: 15)
3′-AGTTGCGCTAGTCTTTCGAAGTACACC-5′,
(SEQ ID NO: 16)
5′-CTGAAAGTCCCCAAGGCTAACAGAA-3′,
(SEQ ID NO: 17)
3′-AGGACTTTCAGGGGTTCCGATTGTCTT-5′,
(SEQ ID NO: 18)
5′-ACGATGATGGTCAAAACATCATCAA-3,
or
(SEQ ID NO: 19)
3′-AATGCTACTACCAGTTTTGTAGTAGTT-5′.

In certain embodiments, the inhibitory nucleic acid molecule comprises a sequence with at least 80%, 85%, 90%, or 95%, particularly at least 90%, 95%, 97%, or 99%, more particularly at least 97% or 99% identity with SEQ ID NO: 12, 13, 14, 15, 16, 17, 18, or 19 (or the RNA equivalent). In certain embodiments, the inhibitory nucleic acid molecule comprises a sequence which is a portion of SEQ ID NO: 12, 13, 14, 15, 16, 17, 18, or 19 (or the RNA equivalent). For example, the inhibitory nucleic acid molecule may comprise a sequence which is SEQ ID NO: 12, 13, 14, 15, 16, 17, 18, or 19 (or the RNA equivalent) wherein the terminal 1, 2, 3, 4, 5, 6, 7, 8, 9, or 10 nucleotides are deleted/absent from the 5′ and/or 3′ end of SEQ ID NO: 12, 13, 14, 15, 16, 17, 18, or 19 (or the RNA equivalent). In certain embodiments, the inhibitory nucleic acid molecule comprises SEQ ID NO: 12, 13, 14, 15, 16, 17, 18, or 19 (or the RNA equivalent) plus the nucleotides (e.g., 1, 2, 3, 4, 5, 6, 7, 8, 9, or 10 nucleotides) at the 5′ and/or 3′ present in SEQ ID NO: 2 or 3 (or the RNA equivalent).

In certain embodiments, the inhibitory nucleic acid molecule is an siRNA or shRNA which comprises:

(SEQ ID NO: 28)
5′-GUCAAGAAGCACAUCUUAAACAUGC-3′,
and
(SEQ ID NO: 29)
3′-GACAGUUCUUCGUGUAGAAUUUGUACG-5′.

In certain embodiments, the inhibitory nucleic acid molecule is an siRNA or shRNA which comprises:

(SEQ ID NO: 30)
5′-AACGCGAUCAGAAAGCUUCAUGUGG-3′,
and
(SEQ ID NO: 31)
3′-AGUUGCGCUAGUCUUUCGAAGUACACC-5′.

In certain embodiments, the inhibitory nucleic acid molecule is an siRNA or shRNA which comprises:

(SEQ ID NO: 32)
5′-CUGAAAGUCCCCAAGGCUAACAGAA-3′,
and
(SEQ ID NO: 33)
3′-AGGACUUUCAGGGGUUCCGAUUGUCUU-5′.

In certain embodiments, the inhibitory nucleic acid molecule is an siRNA or shRNA which comprises:

(SEQ ID NO: 34)
5′-ACGAUGAUGGUCAAAACAUCAUCAA-3′,
and
(SEQ ID NO: 35)
3′-AAUGCUACUACCAGUUUUGUAGUAGUU-5′.

The inhibitory nucleic acid molecules of the instant invention may comprise at least one modification. The inhibitory nucleic acid molecules may comprise modifications to enhance in vivo activity, thermodynamic stability and/or nuclease resistance. In certain embodiments, the inhibitory nucleic acid molecule comprises a modified linkage, modified sugar group, modified base, and/or modified phosphate groups. Exemplary modifications to nucleic acid molecules are provided in U.S. Application Publication No. 20050032733, incorporated by reference. Examples of modifications on the sugar moiety include but are not limited to: locked nucleic acids (LNA), unlocked nucleic acids (UNA), 2″-deoxy, 2′-O-methyl, 2′-fluoro, 2′-methoxyethyl, and 2′-aminoethyl. Examples of modified bases include but are not limited to: pseudouridine, 1-methylpseudouridine, 1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, 2′-O-methyl uridine, hypoxanthine, 2,4-di-fluorotoluene, and dihydrouridine, particularly hypoxanthine, 2,4-di-fluorotoluene, dihydrouridine, 2′-thiouridine, or pseudouridine. Examples of phosphate modifications include but are not limited to: phosphorothioate, boranophosphate, and peptide nucleic acids (PNAs). In certain embodiments, the inhibitory nucleic acid molecules comprise a nuclease resistant modification such as phosphorothioates, locked nucleic acids (LNA), 2′-O-methyl modifications, and/or morpholino linkages.

When an inhibitory nucleic acid molecule is delivered to a cell or subject, the inhibitory nucleic acid molecule may be administered directly or a nucleic acid molecule encoding the inhibitory nucleic acid molecule (e.g., a vector (e.g., expression vector)) may be used. In accordance with the instant invention, nucleic acid molecules encoding at least one inhibitory nucleic acid molecule are provided. In certain embodiments, the nucleic acid molecules encoding at least one inhibitory nucleic acid molecule are contained within a vector, particularly an expression vector. The expression vectors of the instant invention may employ a strong promoter, a constitutive promoter, and/or a regulated promoter. In certain embodiments, the inhibitory nucleic acid molecules are expressed transiently. In certain embodiments, the promoter is cell-type specific (e.g., pancreatic cells). Examples of promoters, particularly promoters well suited for expressing short nucleic acid molecules, are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of vectors for expressing the molecules of the invention include, without limitation, plasmids and viral vectors (e.g., adeno-associated viruses (AAVs), retroviruses, and lentiviruses).

The inhba inhibitor (e.g., inhibitory nucleic acid molecule) may also be contained within a vehicle. In certain embodiments, the vehicle is targeted to the desired site (e.g., cancerous cells (e.g., pancreatic cancer cells, colon cancer cells, liver cancer cells, and cancer cells of the head and neck)). In certain embodiments, the vehicle comprises a CXCR4 targeting moiety. In certain embodiments, the vehicle comprises a CXCR4 antagonist or ligand (e.g., AMD3100, hydroxychloroquine, T140, or antibody 12G5).

In certain embodiments, the vehicle is a nanoparticle, micelle, microparticle, lipid polymer, or hydrogel. In certain embodiments, the inhba inhibitor (e.g., inhibitory nucleic acid molecule) is contained within a nanoparticle. In certain embodiments, the nanoparticle is a polyplex. In certain embodiments, the nanoparticle is a PCX nanoparticle (e.g., Xie et al. (2019) Wiley Interdiscip Rev Nanomed Nanobiotechnol., 11(2): e1528; Li et al. (2012) Angew. Chem., 124:8870-8873; Xie et al. (2020) ACS Nano 14(1):255-271, each incorporated herein by reference). In certain embodiments, the PCX nanoparticle comprises a copolymerization of AMD3100 with hexamethylene bis(acrylamide) and covalently modified with cholesterol (e.g., to improve the in vivo stability).

Compositions comprising at least one inhba inhibitor and at least one carrier (e.g., a pharmaceutically acceptable carrier) are also encompassed by the instant invention. Except insofar as any conventional carrier is incompatible with the variant to be administered, its use in the pharmaceutical composition is contemplated. In certain embodiments, the composition comprises at least one inhibitory nucleic acid molecule and/or at least one nucleic acid molecules encoding at least one inhibitory nucleic acid molecule. In certain embodiments, the composition further comprises a therapeutic agent for treating the disease or disorder.

In accordance with another aspect of the instant invention, methods for inhibiting (e.g., reducing or slowing), treating, and/or preventing a disease or disorder in a subject are provided. In certain embodiments, the method comprises administering an inhba inhibitor described herein to the subject. In certain embodiments, the methods comprise administering to a subject in need thereof a therapeutically effective amount of at least one inhba inhibitor (e.g., an inhibitory nucleic acid molecule and/or nucleic acid molecule encoding an inhibitory nucleic acid molecule) to the subject. The inhba inhibitor may be administered in a composition further comprising at least one pharmaceutically acceptable carrier. In certain embodiment, the methods further comprise administering at least one other/additional therapeutic agent or therapy to the subject (e.g., chemotherapeutic agent, radiation therapy, etc.).

These inhba inhibitors of the instant invention can be used to treat a variety of diseases and disorders including but not limited to: cancers, autoimmune diseases, fibrotic disorders, blood disorders, allergies and allergic diseases, heart failure, neurodegenerative diseases, cachexia, and inflammatory diseases. Examples of specific diseases and disorders include but are not limited to: pancreatic cancer, lung cancer, blood cancers, breast cancer, skin cancer, head and neck squamous cell carcinoma, granulosa cell tumor, colorectal cancer, gastric adenocarcinoma, esophageal squamous cell carcinoma, colon adenocarcinoma, urothelial carcinoma, rheumatoid arthritis, pulmonary fibrosis, kidney fibrosis, liver fibrosis, Alzheimer's disease, fibrodysplasia ossificans progressive, thalassemia major, double heterozygous sickle cell/beta-thalassemia, cardiac dysfunction, cachexia, pancreatitis, allergic asthma, atopic dermatitis, pulmonary alveolar proteinosis, and systemic lupus erythematosus. In certain embodiments, the disease or disorder is cancer, particularly pancreatic cancer, colon cancer, liver cancer, or head and neck cancer. In certain embodiments, the disease or disorder is pancreatic cancer. In certain embodiments, the cancer is pancreatic ductal adenocarcinoma. In certain embodiments, the disease or disorder is cachexia (e.g., cancer-associated cachexia). In certain embodiments, the disease or disorder is metastasis and/or metastatic growth.

In certain embodiments, the methods further comprise measuring activin A levels in the subject. In certain embodiments, the methods further comprise measuring activin A levels in the subject and administering the inhba inhibitor to the subject wherein activin A levels are increased over the activin A levels in a normal subject (e.g., a healthy subject without the disease or disorder (e.g., cancer) to be treated). In certain embodiments, the activin A levels are measured in a tissue (e.g., the tissue with the disease or disorder (e.g., site of cancer (e.g., tumor))). In certain embodiments, the activin A levels are measured in the sera and/or blood. In certain embodiments, the activin A levels are 2 times, 3 times, 4 times, 5 times, 6 times, 7 times, or more, higher in the subject to be treated than the healthy subject. In certain embodiments, the activin A levels in the blood and/or sera are at least 2 ng/ml, 3 ng/ml, 4 ng/ml, 5 ng/ml, 6 ng/ml, or more for treatment.

As explained hereinabove, the compositions of the instant invention are useful for inhibiting (e.g., reducing or slowing), treating, and/or preventing a disease or disorder. A therapeutically effective amount of the composition may be administered to a subject in need thereof. The dosages, methods, and times of administration are readily determinable by persons skilled in the art, given the teachings provided herein. The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, orally, transdermal, pulmonary, rectal, vaginal, intrapulmonary, intraarterial, intrarectal, intramuscular, intrathecal, intracerebral, epidural, intradermal, intracarotid, and intranasal administration. In certain embodiments, the composition is administered directly to the site of cancer or cancerous tissue or organ (e.g., pancreas, colon, liver, and head and neck). In certain embodiments, the composition is administered intraperitoneally.

In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti-oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. (See, e.g., Remington: The Science and Practice of Pharmacy, 21st edition, Philadelphia, PA. Lippincott Williams & Wilkins). The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated. Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity and pharmacokinetics of the molecules in animal models. Various concentrations of pharmaceutical preparations may be administered to mice with transplanted human tumors, and the minimal and maximal dosages may be determined based on the results of significant reduction of tumor size, fibrotic tissue, or serum level of activin A and side effects as a result of the treatment. Appropriate dosage unit may also be determined by assessing the efficacy of the treatment in combination with other standard therapies.

The pharmaceutical preparation comprising the molecules of the instant invention may be administered at appropriate intervals, for example, at least once a day or more until the pathological symptoms are reduced, alleviated, or cured, after which the dosage may be reduced to a maintenance level or eventually ceased. The appropriate interval in a particular case would normally depend on the condition of the patient.

### Definitions

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, or treat a particular injury and/or the symptoms thereof. For example, a “therapeutically effective amount” may refer to an amount sufficient to modulate the pathology associated with pancreatic cancer.

The term “vector” refers to a carrier nucleic acid molecule (e.g., RNA or DNA) into which a nucleic acid sequence can be inserted, e.g., for introduction into a host cell where it may be expressed and/or replicated. An “expression vector” is a specialized vector that contains a gene or nucleic acid sequence with the necessary operably linked regulatory regions needed for expression in a host cell. The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

The following example illustrates certain embodiments of the invention. It is not intended to limit the invention in any way.

### Example

Pancreatic ductal adenocarcinoma (PDAC) accounts for more than 90% of pancreatic cancer. KRAS mutations are present in nearly 95% of PDAC tumors and co-present with TP53 mutation in approximately 70% of PDAC tumors (Cancer Genome Atlas Research Network (2017) Cancer Cell 32:185). PDAC has approximately 10% 5-year relative survival rates and is the third leading cause of cancer-related deaths in the U.S. (Siegel, et al. (2020) Ca-Cancer J. Clin. 70:7). The dismal prognosis pertains to few early diagnostic tools, chemoresistance, and high incidence of comorbidities including cachexia (Hasan, et al. (2019) Oncol. Rev. 13:410; Principe, et al. (2021) Front. Oncol., 11:688377). Therefore, more efforts are needed towards improving PDAC prognosis by identifying prognostic factors for PDAC and their underlying mechanisms (Shi, et al. (2022) Biosci. Rep., 42: BSR20212523).

Inhibin βA (inhba) is a subunit of activins and inhibin A which are members of the transforming growth factor β family (TGF-β). The homodimer of inhibin βA subunits constitutes activin A, and inhibin βA and inhibin PB (INHBB) form activin AB. Inhibin βA also heterodimerizes with inhibin α (INHA) to make inhibin A which antagonizes activin A by competing for Activin receptors (Gold, et al. (2013) J. Pathol., 229:599). Activin A was first discovered from the porcine follicular fluid as an ovarian hormone (Bloise, et al. (2019) Physiol. Rev., 99:739). Activin A acts through its binding to activin type 2 receptors (ACVR2A or ACVR2B), which recruits type 1 receptors to form a receptor complex (Jatzlau, et al. (2023) Commun. Biol., 6:34). Then, intracellular signaling transductions are mediated by two pathways: the canonical pathway, where SMAD3 is phosphorylated and recruits SMAD4 to acquire transcriptional activity, and the noncanonical pathway, where subfamilies of the mitogen-activated protein kinases including p38 activate downstream molecules (Loomans, et al. (2014) Cancers 7:70). SMAD3-dependent signaling is indispensable for the proliferation of ovarian granulosa cells by activin A, and the downstream targets, Inha and Fst, feedback inhibits activin A-mediated proliferation (Park, et al. (2005) J. Biol. Chem., 280:9135; McTavish, et al. (2013) Mol. Cell. Endocrinol., 372:57). The failure of antagonizing activin A has been linked to the spontaneous development of granulosa cell tumors in mice (Looyenga, et al. (2006) Mol. Endocrinol., 20:2848; Myers, et al. (2013) Biol. Reprod., 88:86).

Serum activin A levels are significantly elevated in Stage IV PDAC patients than in healthy individuals (Xu, et al. (2022) Sci. Rep., 12:1659). Higher activin A levels are associated with metastasis and poor survival in PDAC patients and animals (Mancinelli, et al. (2021) Sci. Rep., 11:7986; Zhong, et al. (2019) J. Cachexia, Sarcopenia Muscle, 10:1083). Correspondingly, high tumor SMAD3 expression is associated with tumor size, lymph node metastasis, and poor survival in PDAC patients (Yamazaki, et al. (2014) Lab. Invest., 94:683). However, it was unclear whether activin A suppression would hold therapeutic potentials for PDAC due to, in part, a limited understanding of target cells and/or mechanisms which activin A acts through to promote PDAC.

Cellular heterogeneity is a prominent feature of PDAC and is characterized by the co-existence of heterogenous tumor cells at different subtypes and states and interconvertible activation states of cancer-associated fibroblasts (CAFs) which largely originate from pancreatic stellate cells (PSCs) (Geng, et al. (2021) Front. Cell Dev. Biol., 9:655152; Wandmacher, et al. (2021) Cancers 13:4932; Krieger, et al. (2021) Nat. Commun., 12:5826). CAFs are subtyped by different gene expression patterns and modulate metastasis and immune exclusion (Geng, et al. (2021) Front. Cell Dev. Biol., 9:655152; Lin, et al. (2020) Genome Med., 12:80; Chen, et al. (2021) EBioMedicine 66:103315). High α-SMA-expressing myofibroblastic CAFs (myCAF) form dense fibrosis, prevent metastasis, and promote T cell infiltration in genetically engineered PDAC mice (Ozdemir, et al. (2014) Cancer Cell 25:719; Feldmann, et al. (2021) Gastroenterology 160:346). However, inflammatory CAFs (iCAFs), which highly express inflammatory cytokines including IL-6, decrease T cell infiltration and tumor cell death, promoting PDAC growth (Garg, et al. (2018) Gastroenterology 155:880). Therefore, PDAC progression is accounted for by not only tumor cell proliferation but also their reciprocal interaction with CAFs.

Here, the expression of inhibin βA protein is investigated in normal pancreas, chronic pancreatitis, premalignant pancreatic intraepithelial neoplasia (PanIN), and PDAC in humans. In orthotopic PDAC mice, the therapeutic potentials of tumor activin A on tumor growth was characterized and tumor-promoting roles of activin A in vivo and in vitro were addressed.

### Materials and Methods

### Cell Culture

Acinar 266-6 (ATCC, CRL-2151), KPC1245, 8060, and 8069 cell lines were cultured in RPMI1640 supplemented with 10% fetal bovine serum and 1% Antibiotic-Antimycotic under a 5% CO₂ incubator at 37° C. All cell lines were used at the passages between 8 to 13. Conditioned media were collected from KPC8069 (KPC-CM) at 100% confluence and filtered with a 0.02 μm pore size.

### Pancreatic Stellate Cell (PSC) Isolation and Culture

The isolation and culture of PSCs were performed as described (Feldmann, et al. (2021) Gastroenterology 160:346). Briefly, normal mouse pancreatic tissues (n=5) were minced and digested in Gey's balanced salt solution (GBSS) containing 0.3% bovine serum albumin, 0.05% collagenase P solution, 0.02% pronase, and 0.1% DNase. After centrifugation, the pellet was resuspended in 4.75 mL GBSS/0.3% BSA and 4 mL 28.7% Histodenz™ (Sigma, D2158) in GBSS solution. After adding a layer of 3 mL GBSS/0.3% BSA, the mixture was centrifuged, and the white layer above the interface was collected and cultured in the media of 50% DMEM-F12 and 50% DMEM supplemented with 20% FBS and 1% antibiotic-antimycotics. To collect the conditioned media (CM) from the cells, stellate cells were stimulated with activin A for 24 hours, and then fresh media were added after washing twice with PBS and collected as CM after additional 24 hours.

### Cell Growth and Invasion Assay

Cell growth experiments were performed by seeding cells at 3000 cells in a 6-well plate in the presence or absence of designated treatments. After 48-72 hours, the cells were fixed with 10% formaldehyde, washed with PBS three times, and stained with 0.1% crystal violet in PBS for 10 minutes. After washing with distilled water, the wells were air-dried and eluted using 50% DMSO in PBS, and the absorbance was measured at 540 nm. Chemoinvasion activity was examined using 24-well plates with transwell inserts coated with Matrigel® (Corning, 354480). 5×10⁵ cells were seeded on the transwell inserts in 1% FBS-RPMI, and the inserts were placed in 24-well plates with 10% FBS-RPMI. After 24 hours, the invaded cells were fixed and stained with 0.1% crystal violet. Noninvaded cells were scraped off from the wells. Relative invasion abilities were compared by calculating cell-invaded areas using ImageJ (NIH software, Fiji v2.3.1).

### Real-Time (RT) PCR

Total RNAs were collected using TRIzol® reagent and synthesized into cDNAs with the high-capacity RNA-to-cDNA kit. RT-PCR was performed on the CFX96 Real-Time PCR Detection System (Bio-Rad Laboratories, 185-5096). The target genes were normalized with 18s, and the relative expression was calculated using the 2^{-ΔΔCT}. The primers used are listed in Table 1.

TABLE 1
List of sequences for primers.

| Gene | Primer Sequence |
|---|---|
| Inhba | Mm00434339_m1 (TaqMan ® probe) |
| Muc5b | S: 5′-GCATCATCAACAGTGCAACG-3′ (SEQ ID NO: 36) |
| | AS: 5′-GCAGAAACACTCGCAGTCA-3′ (SEQ ID NO: 37) |
| Muc1 | S: 5′-CCTACCATCCTATGAGTGAATACC-3′ (SEQ ID NO: 38) |
| | AS: 5′-GACTGCTACTGCCATTACCTG-3′ (SEQ ID NO: 39) |
| Muc4 | S: 5′-CAGCCTCTCCAAGAAATGTAGT-3′ (SEQ ID NO: 40) |
| | AS: 5′-GACAAGTTAGTCCTGACATCCC-3′ (SEQ ID NO: 41) |
| Muc5ac | S: 5′-CCACTTTCTCCTTCTCCACACC-3′ (SEQ ID NO: 42) |
| | AS: 5′-GGTTGTCGATGCAGCCTTGCTT-3′ (SEQ ID NO: 43) |
| Acvr1a | S: 5′-CCATTGAAGGGCTCATCACCAC-3′ (SEQ ID NO: 44) |
| | AS: 5′-CCGTTCTTGTACCAGGAAAGG-3′ (SEQ ID NO: 45) |
| Acvr1b | S: 5′-TTCTTCCCCCTTGTTGTCCTC-3′ (SEQ ID NO: 46) |
| | AS: 5′-ACAGGTGTAGTTGGTCTGTAGG-3′ (SEQ ID NO: 47) |
| Acvr1c | S: 5′-AGACGGTGATGCTGAGACACGA-3′ (SEQ ID NO: 48) |
| | AS: 5′-GACCATTCCAGCCACAGTCACT-3′ (SEQ ID NO: 49) |
| Acvr2a | S: 5′-TCATACCTGCATGAGGATGTG-3′ (SEQ ID NO: 50) |
| | AS: 5′-CCAAGCCAAAGTCAGCCA-3′ (SEQ ID NO: 51) |
| Acvr2b | S: 5′-CCTGGCTGTTCGGTTTGAG-3′ (SEQ ID NO: 52) |
| | AS: 5′-GCGTCTCTCTGGAAGTTGATG-3′ (SEQ ID NO: 53) |
| RN18S | Mm03928990_g1 (TaqMan ® probe) |

### Animal and Orthotopic Implantation

The animals were given free access to food and water and kept in Comparative Medicine facilities. 12-week-old C57BL/6 male mice were used to generate an orthotopic PDAC model by implanting 5×10⁴ KPC8069 cells. The sham group received the same operation of procedure except for cell implantation. Post-operative monitoring was carried out for 3 days (D3), and the orthotopic mice were divided into 3 groups on D4: treatment with no siRNA (tumor only, TO), scramble siRNA (Sc-si), or inhba siRNA (Inhba-si). Body-weight and food intake were measured daily.

Administration of siRNA-PCX Nanoparticles

siRNA-PCX nanoparticles were prepared as described (Xie, et al. (2020) ACS Nano, 14:255). Polyplexes (2.5 mg kg⁻¹ siRNA, 5 mg kg⁻¹ PCX) were intraperitoneally injected into the orthotopic mice on D4, 11, 13, and 15. The sequences for the scramble and inhba siRNA are listed in Table 2. For biodistribution, Cy5.5-labeled siRNA was mixed with PCX polyplexes and given to the orthotopic mice at D11. After 24 hours, tissues were harvested for ex vivo fluorescence imaging using Xenogen IVIS® 200 (675 or 720 nm). Tumors were encapsulated in Tissue-Tek® O.C.T. Compound, sectioned using cryostat at 8 μm, and stained with DAPI. In situ distribution of Cy5.5 fluorescence was imaged with the confocal microscope.

TABLE 2
List of sequences for siRNA. The scrambled negative control DsiRNA
is a nonsilencing, negative control Dicer-substrate siRNA (DsiRNA)
DsiRNA that does not recognize any sequences in human, mouse, or
rat transcriptomes.

| Gene | siRNA sequences |
|---|---|
| #1 Inhba | 5′-AACGCGAUCAGAAAGCUUCAUGUGG-3′ (SEQ ID NO: 30) |
| DsiRNA | 3′-AGUUGCGCUAGUCUUUCGAAGUACACC-5′ (SEQ ID NO: 31) |
| #2 Inhba | 5′-CUGAAAGUCCCCAAGGCUAACAGAA-3′ (SEQ ID NO: 32) |
| DsiRNA | 3′-AGGACUUUCAGGGGUUCCGAUUGUCUU-5′ (SEQ ID NO: 33) |
| #3 Inhba | 5′-ACGAUGAUGGUCAAAACAUCAUCAA-3′ (SEQ ID NO: 34) |
| DsiRNA | 3′-AAUGCUACUACCAGUUUUGUAGUAGUU-5′ (SEQ ID NO: 35) |
| #4 Inhba | 5′-GUCAAGAAGCACAUCUUAAACAUGC-3′ (SEQ ID NO: 28) |
| DsiRNA | 3′-GACAGUUCUUCGUGUAGAAUUUGUACG-5′ (SEQ ID NO: 29) |
| Scrambled | Cat. # 51-01-19-09, Integrated DNA Technologies |
| negative | (species: Human, Mouse, Rat) |
| control | |
| DsiRNA | |

### Human Pancreatic Tissue Microarray

PDAC tissue microarrays (TMA) (US Biomax, Inc.) consisted of 60 cases of PDAC and 9 normal cases (3/case). Pancreatitis TMA had 14 cases of PanIN, 6 pancreatitis, and 4 PDAC (2/case). TMA slides were used for DAB staining.

### Evaluation of Serum Cytokines

Serum levels of activin A, inhibin A, IL-6, and GDF15 were quantified using commercially available ELISA kits [Activin A and inhibin A ELISA kits (Ansh Labs, AL-110 and AL-123), IL-6 ELISA (Invitrogen, BMS603-2), and GDF15 Quantikine™ ELISA (R&D Systems, MGD150)].

### Immunohistochemistry (IHC) Assay

Tissue slides with 5 μm thickness were subject to hematoxylin/eosin (H&E), Masson's trichrome staining, or picrosirius red solution (PSR) for histological examination. DAB (3, 3′-diaminobenzidine) and immunofluorescence assays were performed as described (Xu, et al. (2022) Sci. Rep., 12:1659.). All the images were taken with the EVOS™ M7000 Imaging System (Invitrogen, AMF700). Antibody specificity was tested with positive control tissues. Antibody information: Inhibin βA (Rabbit, 1:50, Wylie Vale), SOX9 (Rabbit, 1:1000, Cell Signaling Technology 82630S), Cytokeratin 19 (Rat, 1:100, Development Studies Hydridoma Bank TROMA-III-c), Smad3 (Rabbit, 1:1000 or 1:50, Cell Signaling Technology 9523S), α-SMA (Rabbit, 1:50, Cell Signaling Technology 19245T), Acvr2A (Rabbit, 1:50, Millipore Sigma HPA046997), Acvr2B (Rabbit, 1:50, Invitrogen PA5-111122), Inhibin α (Rabbit, 1:50, Wylie Vale), Bax (Rabbit, 1:50, Cell Signaling Technology #2772), PD-L1 (Rabbit, 1:50, R&D Systems AF1019), Prrx1 (Rabbit, 1:100, Thermo Fisher PA5-106700), CD3 (Rabbit, 1:50, Cell Signaling Technology 78588T), CD8 (Rabbit, 1:50, Cell Signaling Technology 98941T), Amylase (Rabbit, 1:50, Cell Signaling Technology 3796S), p-p38 (Rabbit, 1:1000, Cell Signaling Technology 9211S), p38 (Rabbit, 1:1000, Cell Signaling Technology 9212S), p-Smad3 (Rabbit, 1:1000, Rockland 600-401-919), E-Cadherin (Rabbit, 1:50, Cell Signaling Technology 14472S), N-Cadherin (Rabbit, 1:50, Cell Signaling Technology 13116S), CFTR (Rabbit, 1:50, Novus Biologicals NB300-511), a/0-Tubulin (Rabbit, 1:1000, Cell Signaling Technology 2148S), Anti-rabbit IgG (tRP, 1:2000, Cell Signaling Technology 7074), Anti-mouse IgG (HRP, 1:2000, Cell Signaling Technology 7076), Anti-rabbit IgG (Biotinylated, 1:200 or 1:400, Vector Laboratories BA-1000-1.5), Anti-rat IgG (Biotinylated, 1:200 or 1:400, Vector Laboratories BA-9400-1.5), Anti-mouse IgG (Alexa Fluor® 488, 1:100-200, Invitrogen A-11001), Anti-rabbit IgG (Alexa Fluor® 488, 1:100-200, Invitrogen A-11008), Anti-rat IgG (Alexa Fluor® 568, 1:100-200, Invitrogen A-11077), and Anti-goat IgG (Alexa Fluor® 488, 1:100, Invitrogen A-11055).

### Immunoblotting

Tissue lysates were prepared using RIPA buffer supplemented with protease and phosphatase inhibitors. Proteins (20 g) were separated on 12.5% SDS polyacrylamide gel, transferred to PVDF membranes, and incubated with primary and secondary antibodies. Protein bands were captured by a c500 imaging system (Azure Biosystems). The band intensities were determined using ImageJ software.

### Statistics

Graphs were generated by GraphPad Prism 9.3.1 software, and in vivo data were presented as mean±standard error of the mean (SEM). The one-way analysis of variance (ANOVA) with Tukey's post-hoc test was performed followed by unpaired two-tailed Student's t-tests. The number of each group for animal experiments started with 5/group except for the sham group (n=4). During the experimental period, one from the TO group died on D15. Then, the sample size was justified using G*power software v3.1 with post-hoc analysis using pancreas weights. The calculated power was 0.99. In vitro data were presented as mean±standard deviation (SD), and statistical significance was determined by paired t-test. P-value was expressed as follows; *P<0.05; **P<0.01; ***P<0.001; ****P<0.0001; n.s., not significant.

### Results

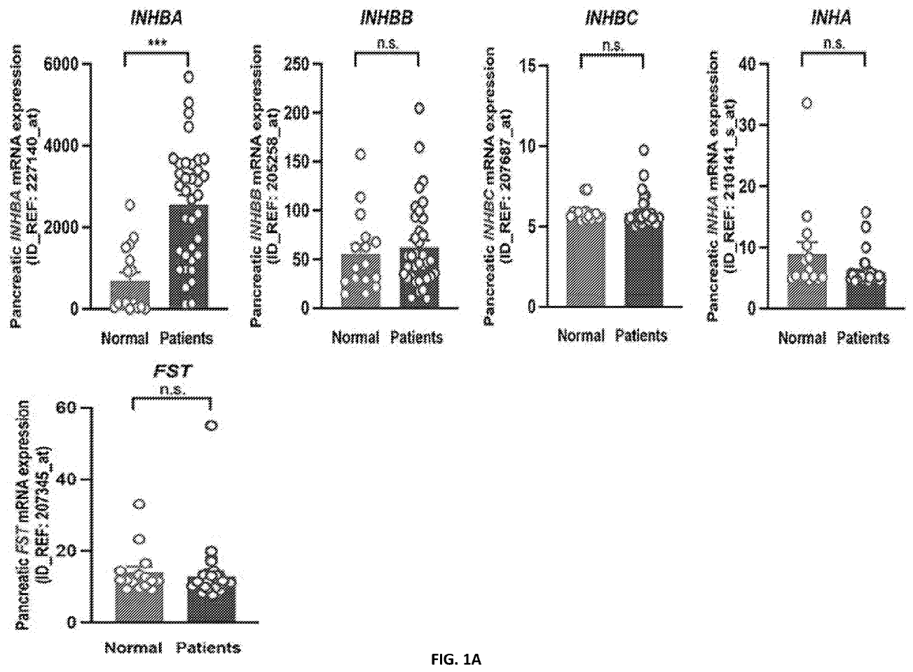

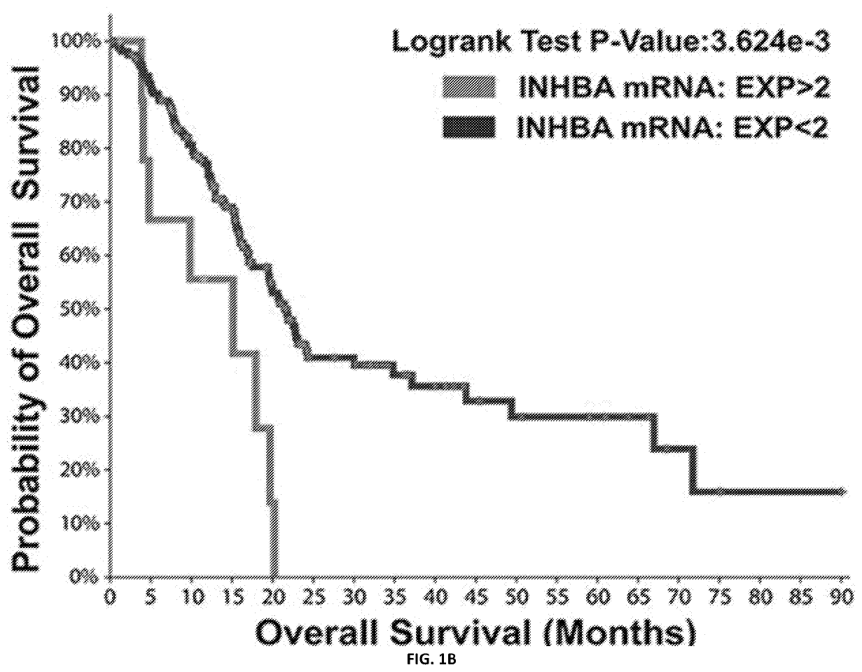

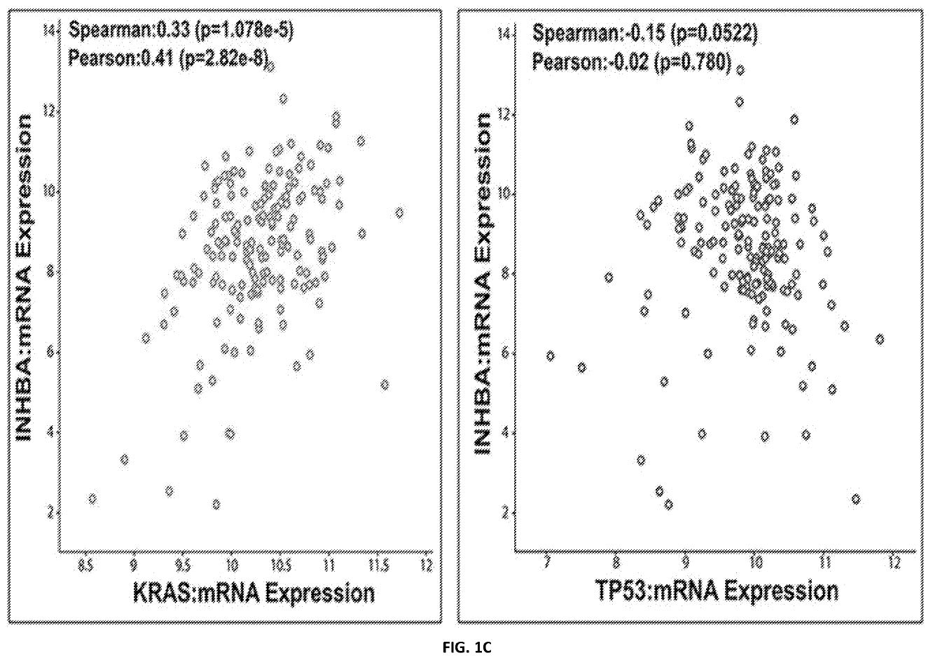

Inhibin βA is a subunit for activin isoforms. First, the expression of activins and inhibin A subunits was characterized in normal human pancreas and pancreatic tumors by accessing the Gene Expression Omnibus database using the accession number GSE16515. INHBA expression was significantly upregulated in tumors than in normal pancreas, but expressions for other isoforms, INHBB and INHBC, remained unchanged (FIG. 1A). The expressions of INHA and FST genes were downregulated and statistically non-significant, respectively (FIG. 1A). Additionally, ACVR2A expression was similar between normal pancreas and tumor, whereas ACVR2B was downregulated in the tumors. Further, expressions for TNF, GDF15, and IL6 were compared, which are recognized as prognostic cytokines for PDAC along with activin A (Rupert, et al. (2021) J. Exp. Med., 218:e20190450; Zhao, et al. (2020) Cell Death Dis., 12:649). Unlike INHBA, expressions of TNF, GDF15, and IL6 genes were not statistically significant between normal pancreas and tumors.

Next, the correlations between INHBA expression and PDAC survival rates or expression of oncogene KRAS and TP53 were examined in patients using The Cancer Genome Atlas-Pancreatic Adenocarcinoma on cBioPortal (cbioportal.org). Pancreatic INHBA expression is correlated with poor PDAC survival (FIG. 1B). INHBA expression was positively correlated with KRAS expression and showed a downward trend as TP53 expression was increased in tumors (FIG. 1C). Further, IHC analysis with TMA identified that the expression of inhibin βA protein was induced in atrophic acinar cells and was intensively detected in PanIN lesions and PDAC cells. Consistently, inhibin βA was significantly induced in atrophic acinar cells than in normal acinar cells and overexpressed in PDAC cells on two KPC tumors. Also, stromal cells express inhibin βA in human and mouse PDAC.

Nearly 95% of PDAC tumors harbor KRAS mutations and 70% of PDAC tumors have KRAS and TP53 mutations (Cancer Genome Atlas Research Network (2017) Cancer Cell, 32:1). Different cancer subtypes with different secretory states and the presence of non-tumor cells such as fibroblasts and immune cells constitute intratumor heterogeneity of PDAC (Krieger, et al. (2021) Nat. Commun., 12:5826; Cros, et al. (2018) Pathobiology 85:64).

Notably, preclinical mouse models of pancreatic ductal adenocarcinoma (PDAC) that mimic human pancreatic cancer provide a model for assessing the efficacy of treatments in humans. The KPC (LSL-Kras^{G12D/+};LSL-Trp53^{R172H/+}; Pdx-1-Cre) mouse model is a mouse model of PDAC which reflects the biology of human pancreatic cancer (Lee et al. (2016) Curr. Protocols Pharmacol., 73:14.39.11-14.39.20). Mutations in the KRAS and TP53 genes are commonly observed in PDAC tumors from patients. The spontaneous pancreatic tumors produced by the KPC mouse model recapitulate the clinical, histopathological and genomic features of human PDAC (Torres, et al. (2013) PLoS ONE 8(11):e80580). The tumor-derived cell lines KPC8069 can be generated as described (Torres, et al. (2013) PLoS ONE 8(11):e80580).

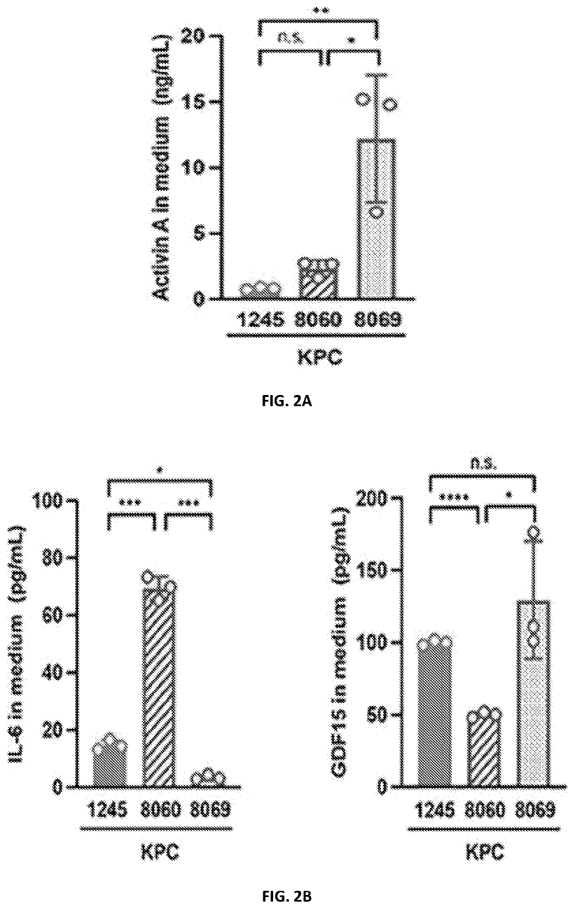

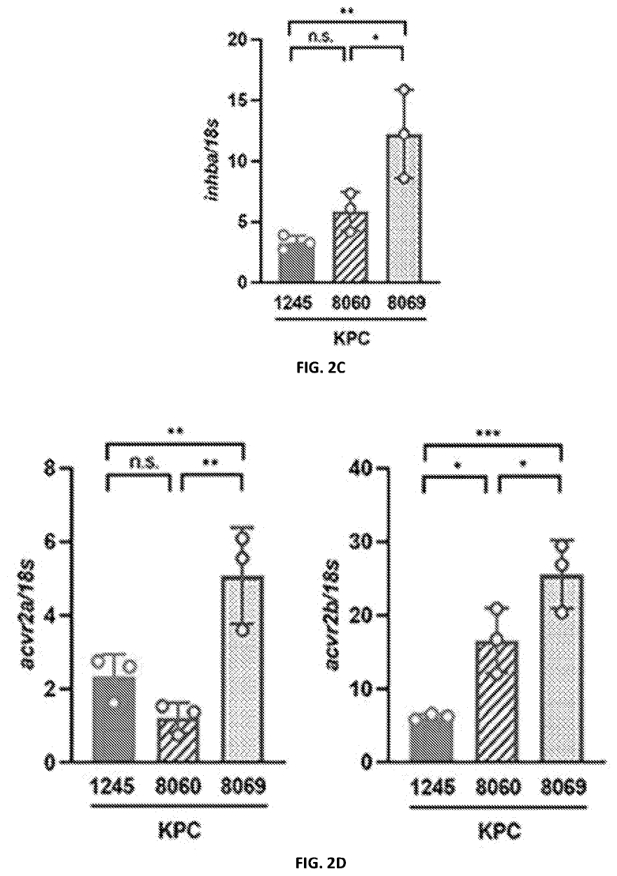

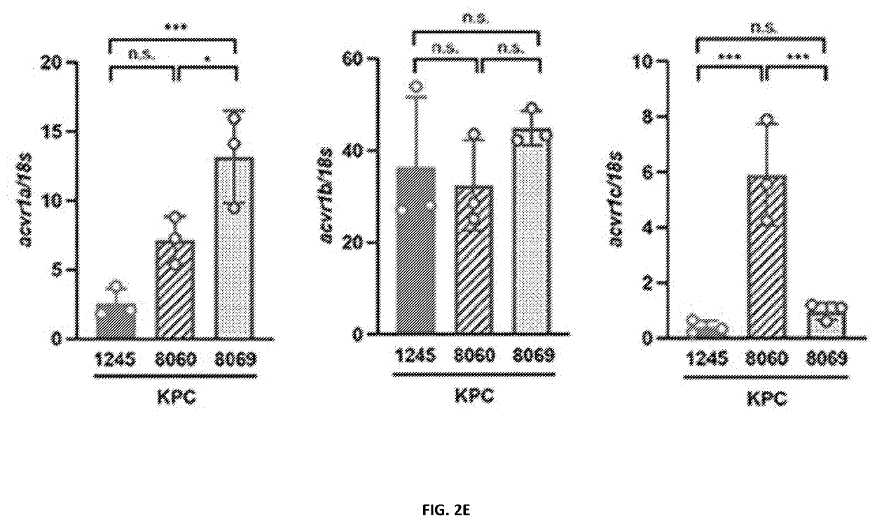

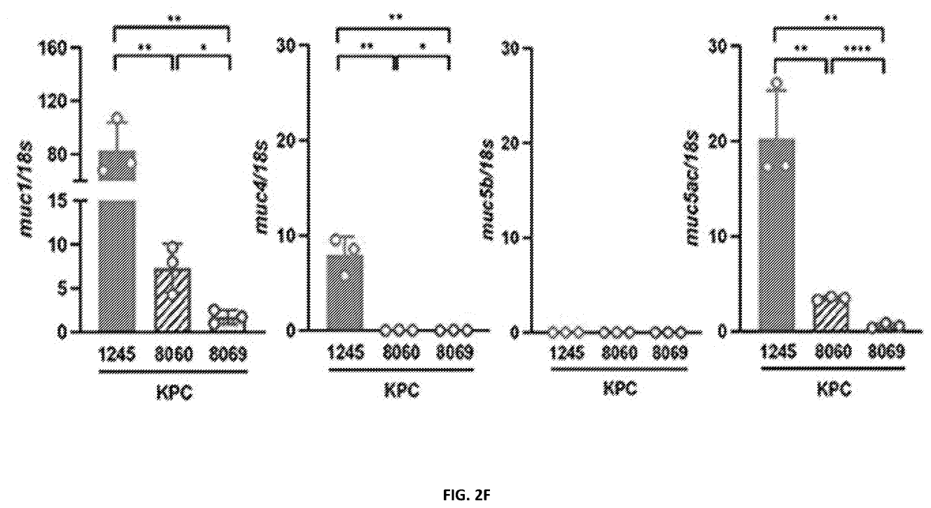

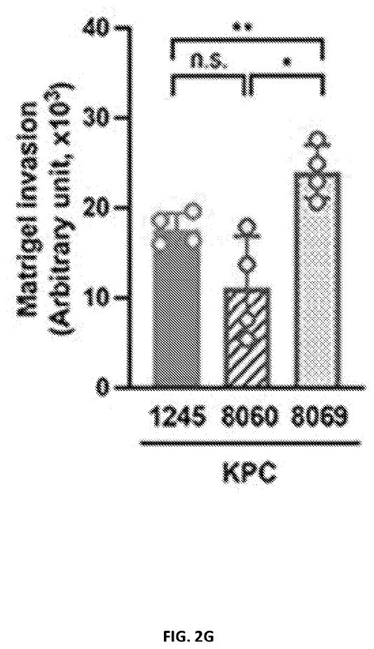

Here, mouse PDAC cell lines harboring Kras^{G12D} and Trp53^{R172H} mutations (KPC), KPC1245, 8060, and 8069, were compared in the secretion of PDAC prognostic cytokines. KPC8069 had significantly higher secretion of activin A when compared to KPC1245 and 8060 (FIG. 2A). IL-6 levels were the highest in the media from KPC8060 and lowest in KPC8069 (FIG. 2B). GDF15 levels were statistically higher in the media from KPC8069 and 1245 than in KPC8060 (FIG. 2B). Different gene expression levels for inhba and activin receptors were observed across KPC cell lines. KPC8069 showed the highest expression levels of genes for Inhba, Acvr2a, Acvr2b, and Acvr1a (FIG. 2C-2E). Acvr1b expression was similar among KPC cell lines, but Acvr1c was the highest in KPC8060 (FIG. 2E).

Mesenchymal N-cadherin is implicated to induce stemness properties of cancer cells and metastasis through epithelial-to-mesenchymal transition and correlates with poor PDAC survival (Luu, T. (2021) Front. Oncol., 11:646399; Loh, et al. (2019) Cells 8:1118; Sommariva, et al. (2020) Cells 9:1040). Next, the correlation between the expression of prognostic cytokines and invasion in KPC cell lines was examined. Epithelial cadherin (E-cadherin) protein was abundantly detected in KPC1245 but less in KPC8060 and 8069. Conversely, N-cadherin was enriched in KPC8060 and 8069 and less detected in KPC1245 cells. All KPC cell lines were negative with amylase but positive with cystic fibrosis transmembrane conductance regulator (CFTR), a ductal cell marker. Coincidently, the expression of genes for mucins (Muc1, 4, and 5ac), commonly referred to as malignant epithelial cell markers (Thompson, et al. (2021) Clin. Cancer Res., 27:6787), was statistically higher in KPC1245, whereas KPC8069 had the lowest expression levels of the genes when normal pancreatic tissue was used as reference expression (FIG. 2F). The Muc5b gene was significantly downregulated in all the KPC cell lines when compared to normal pancreatic tissue (FIG. 2F). In the Matrigel® invasion assay, KPC8069 exhibited faster invasion and migration than KPC1245 and 8060, but no statistical difference between KPC1245 and 8060 was observed (FIG. 2G).

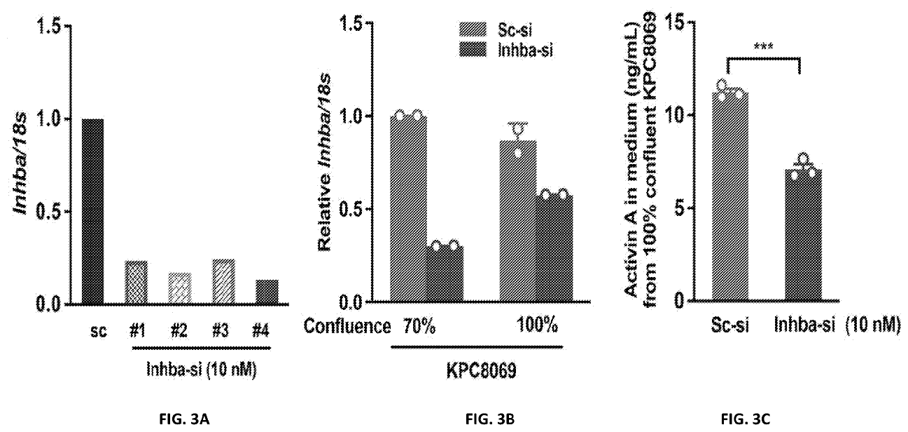

Finally, the inhibitory effects of four different inhba siRNAs were evaluated in KPC8069 because of high activin A expression. All the tested inhba siRNAs at 10 nm showed nearly 80% inhibition on the Inhba expression in KPC8069 (FIG. 3A). Further testing and experiments with #4 inhba siRNA was pursued. The inhba siRNA consistently reduced Inhba expression by approximately 70% and 50% at 70 and 100% confluence, respectively (FIG. 3B). Activin A secretion was also hindered by the inhba siRNA in KPC8069 cells (FIG. 3C).

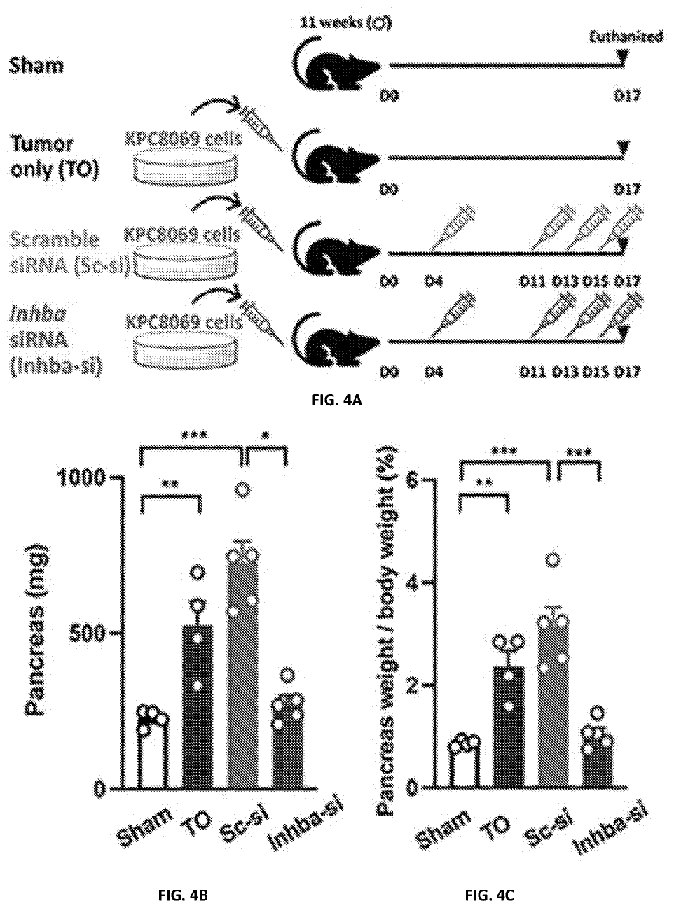

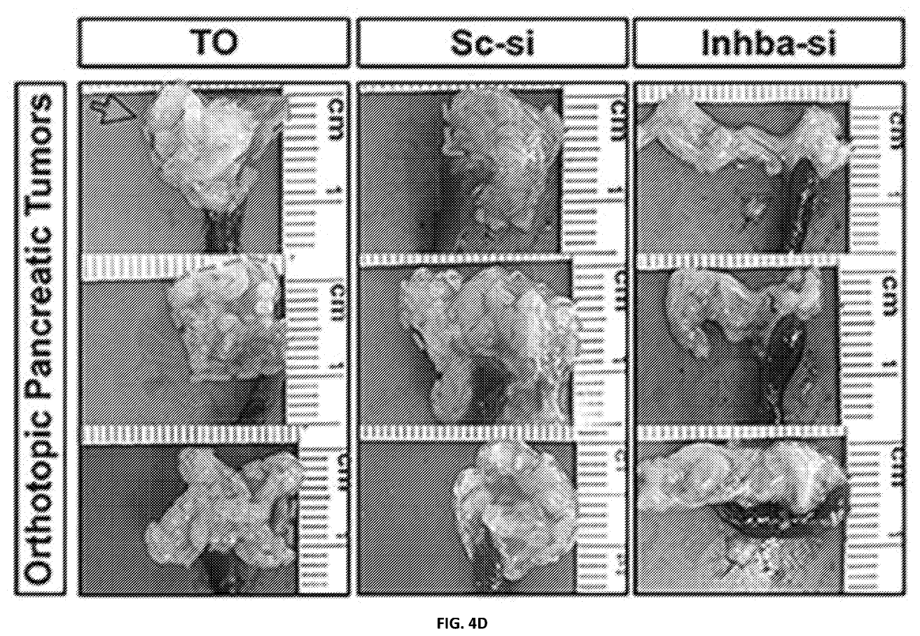

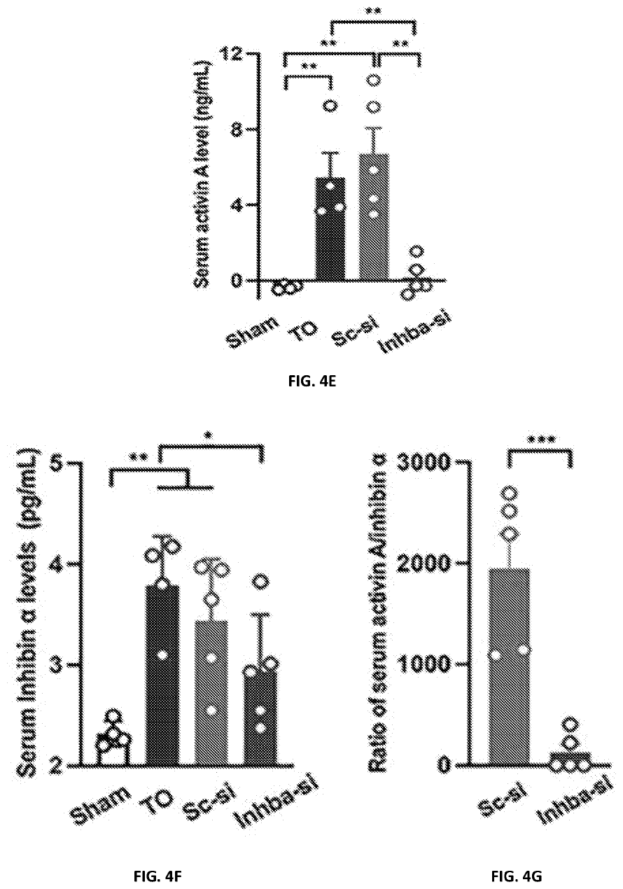

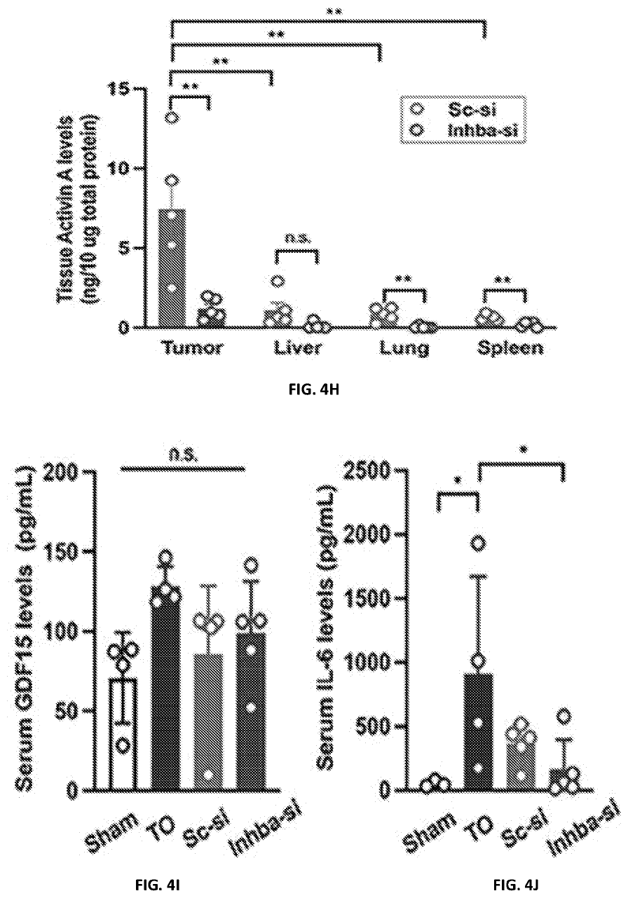

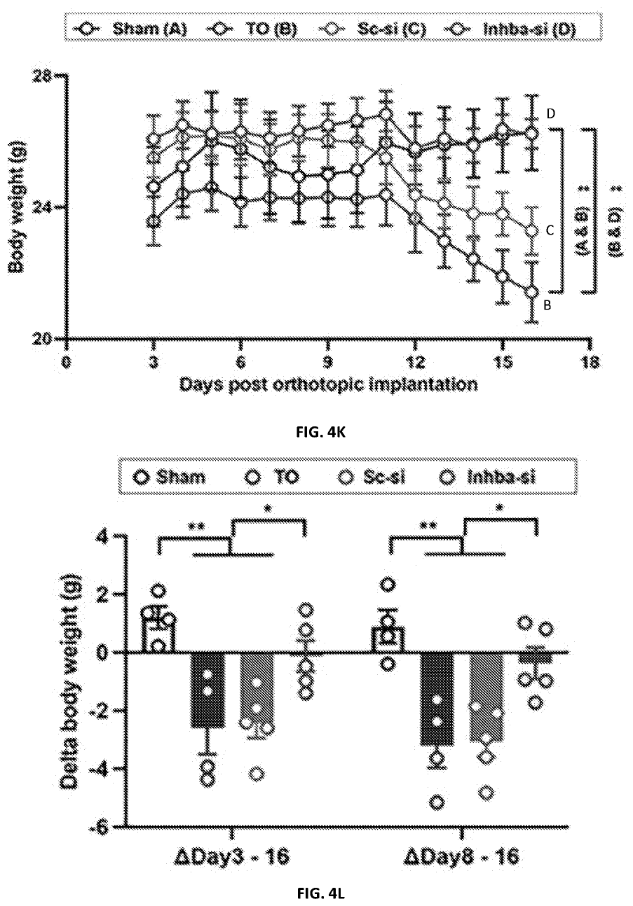

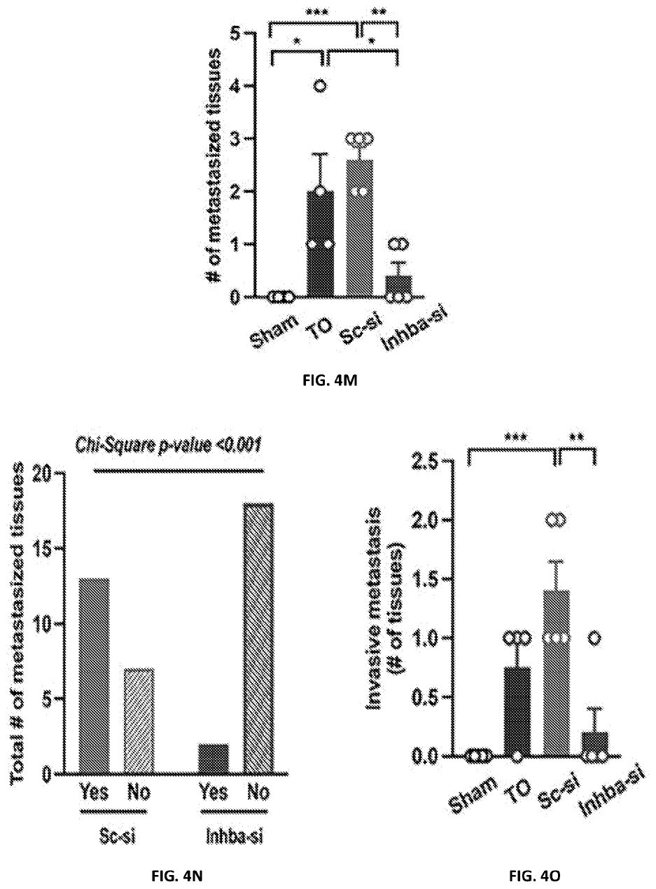

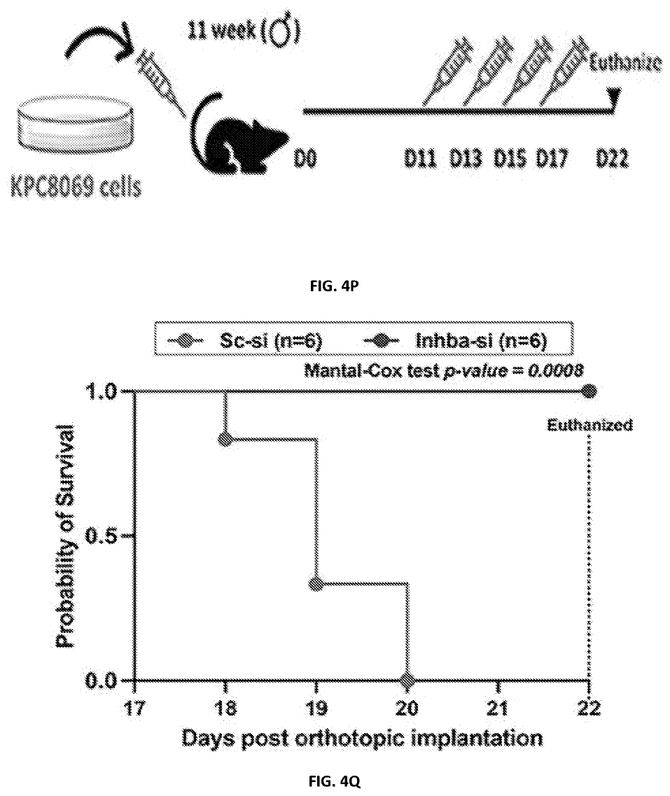

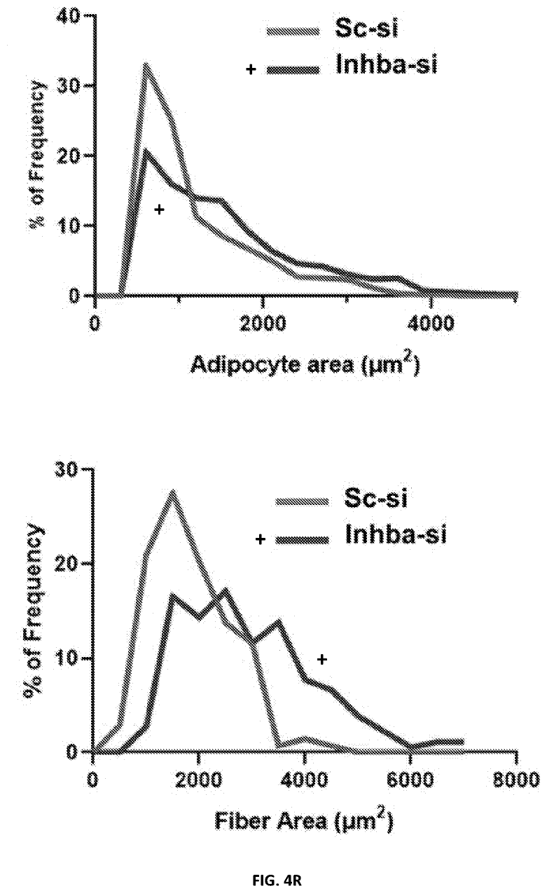

Next, it was examined whether suppressing tumor activin A would retard tumor growth by employing an orthotopic PDAC model. FIG. 4A provides a schematic of the experimental design. The siRNA was packaged with cholesterol-modified polymeric CXCR4 inhibitor (PCX) for tumor-targeted delivery during tumor growth (Xie, et al. (2020) ACS Nano 14:255). Because the knockdown efficiency of inhba siRNA was influenced by cell confluence (FIG. 3B), the initial siRNA injection was on day 4 (D4) of implantation and three injections were made every other day from D11 as most of the pancreas became tumor at D12 as detected by ultrasound imaging. As shown in FIGS. 4B and 4C, significant increases in the pancreas weight and pancreas weight corrected for body weight were observed in both TO and Sc-si groups. FIG. 4D shows the gross appearance of tumors formed in the groups. Serum activin A and inhibin A were considerably elevated in TO and Sc-si groups when compared to the Sham group (FIGS. 4E, 4F). The ratio of activin A to inhibin A showed that circulating activin A was dominantly elevated in the model with a 2000-fold increase in the Sc-si group (FIG. 4G), indicating that the antagonistic role of inhibin A against activin A would be negligible. Furthermore, tissue activin A level was sevenfold higher in tumors than in other organs in the Sc-si group (FIG. 4H). However, no differences in serum GDF15 were found between groups, and serum IL-6 was greatly increased in the TO group (FIGS. 4I, 4J). There was no statistical difference between TO and Sc-si groups unless otherwise stated. Contrastingly, inhba siRNA significantly retarded orthotopic tumor growth as demonstrated by smaller pancreas weights and tumor size in the gross images (FIGS. 4B-4D). Serum activin A and inhibin A levels in the inhba-si group were similar to the Sham group (FIGS. 4E, 4F). The relative ratio of serum activin A to inhibin A and tumor activin A levels were remarkably reduced when compared to the Sc-si group (FIGS. 4G, 4H). Serum IL-6 levels were also significantly reduced in the inhba-si group than in the TO group.

The average body weight started to decline from D12 in both TO and Sc-si groups. Until D16, both TO and Sc-si groups lost 3.5 g on average, which largely happened between D8 and D16 (FIGS. 4K, 4L). However, inhba siRNA prevented weight loss (FIGS. 4K, 4L). Furthermore, significant size reductions in epididymal adipocytes and quadriceps skeletal muscle fiber were mitigated by inhba siRNA treatment (FIG. 4R).

Reduced tumor size by inhba siRNA correlated with less metastasis. A post-mortem examination was performed to identify small nodules on the tissue surface and to test tissue hardness on four nearby tissues: the spleen, liver, kidney, and intestinal lumen. Nodules were found on 8 out of 16 tissues from the TO group (n=4), 13 out of 20 tissues from the Sc-si group (n=5), and 2 out of 20 tissues from the inhba-si group (n=5) (FIG. 4M and Table 3). A chi-square test between groups revealed that there is no statistical difference between TO and Sc-si groups, whereas the inhba-si group had a significantly reduced number of metastasized tissues when compared to TO and Sc-si groups (FIG. 4N and Table 3). Furthermore, it was investigated how many tissues were invasively metastasized with H&E staining (FIG. 4O). TO group had invasive metastasis in 100% of the livers (Table 4). The Sc-si group also had metastasis in 100% of the livers, 33% of the kidneys, and 100% of the intestine. However, the inhba-si group had invasive metastasis in only 40% of the livers. Metastasized cancer cells in the liver were confirmed through IHC for CK19.

TABLE 3
Chi-square test for tissue metastasis

| | Metastasis (nodules) | | |
|---|---|---|---|
| Group | Yes | No | p-value |
| TO | 8 | 8 | ns |
| Sc-si | 13 | 7 | |
| TO | 8 | 8 | <0.05 |
| Inhba-si | 2 | 18 | |
| Sc-si | 13 | 7 | <0.001 |
| Inhba-si | 2 | 18 | |

TABLE 4
Tissues with nodules and H&E-confirmed invasive metastasis.

| | Sham (n = 4) | Tumor (n = 4) | Sc-si (n = 5) | Inhba-si (n = 5) |
|---|---|---|---|---|
| | Tissues with nodules | | | |
| Liver | 0 | 3 (75%) | 5 (100%) | 2 (40%) |
| Spleen | 0 | 2 (50%) | 4 (80%) | 0 |
| Kidney | 0 | 2 (50%) | 3 (60%) | 0 |
| Intestine | 0 | 1 (25%) | 1 (20%) | 0 |
| | Invasive metastasis (H&E confirmed) | | | |
| Liver | 0 | 3 (100%) | 5 (100%) | 1 (50%) |
| Spleen | 0 | 0 | 0 | 0 |
| Kidney | 0 | 0 | 1 (33%) | 0 |
| Intestine | 0 | 0 | 1 (100%) | 0 |

It was then determined whether inhba knockdown increases survival rates in the model. To avoid tumor size-related contribution to survival rates, inhba siRNA was injected every other day from D11 to D17 (FIG. 4P), given that most of the pancreas became tumors at D11 (FIG. 4B). In the Sc-si group, all animals died by D20, whereas no death was observed in the inhba-si group until D22 (FIG. 4Q). The average body weight of the Sc-si group was dramatically decreased, and the mice in the inhba-si group maintained their body weights until D22 (FIG. 4S) with no changes in food intake observed (FIG. 4T). Weight loss in the model would be mediated independently of food intake.

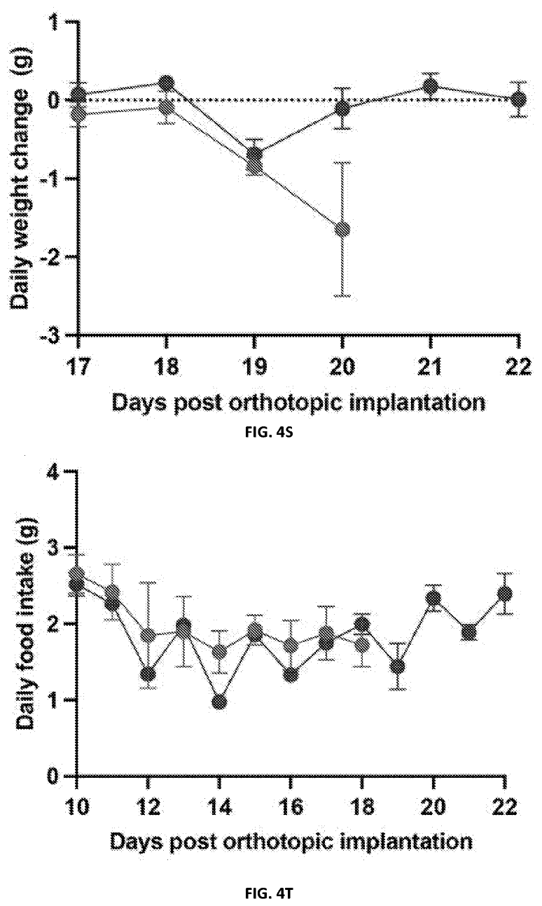

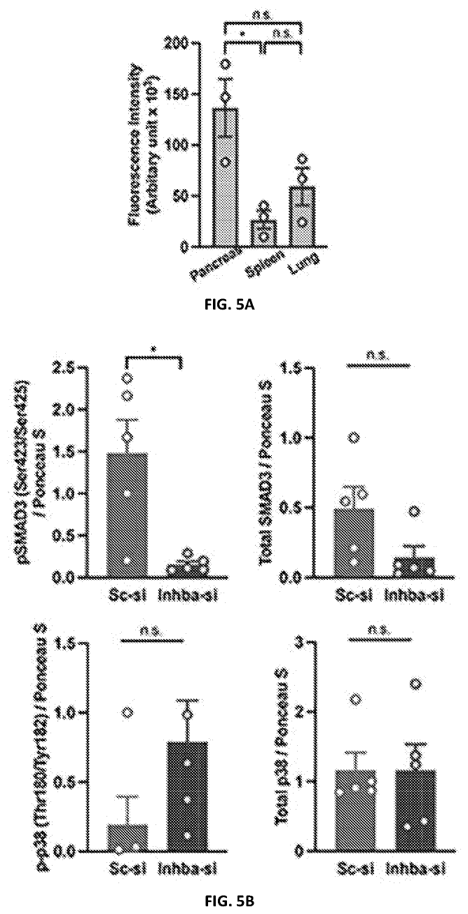

The tumor-targeted siRNA delivery of PCX has been characterized (Xie, et al. (2020) ACS Nano 14:255). PCX biodistribution in this model was confirmed by tagging PCX with Cy5.5-labeled scramble siRNA. After 24 hours of injection, the accumulation of Cy5.5 fluorescence was imaged in different tissues such as the heart, lung, pancreatic tumor, kidney, liver, spleen, skeletal muscles, and adipose tissues. Cy5.5 fluorescence confirmed abundant accumulation in tumor tissues, followed by the lung and spleen (FIG. 5A). Confocal imaging further showed that Cy5.5-labeled siRNA was distributed on the entire surface of frozen tumor sections. The analysis of tumor histology was performed to examine the impact of activin A suppression on PDAC tumors. H&E showed that tumors from the Sc-si group exhibit complete loss of acinar cells but the presence of abnormal cells. A significant accumulation of fibrosis was detected. Masson's Trichrome staining identified the significant accumulation of collagen type 1 in the tumors from the Sc-si group. The local invasion of cancer cells and accumulation of fibroblasts was confirmed through IHC staining of CK19 as a cancer cell marker and α-SMA as an activated fibroblast marker. In addition, the occurrence of tumor-associated acinar-to-ductal metaplasia (ADM) and a significant accumulation of α-SMA-positive fibroblasts around the tumor-associated ADM regions was found. However, inhba siRNA restrained the local invasion of cancer cells, and the majority of the sections from the inhba-si group preserved acinar cells. Although α-SMA-positive fibroblasts were detected around CK19-positive cells, less fibrosis accumulation and collagen type 1 deposition were observed in the tissue sections. Also, less accumulation of α-SMA-positive fibroblasts near tumor-associated ADM regions from the inhba-si group was found.

Tumor SMAD3 correlated with poor PDAC outcomes such as tumor size and metastasis in PDAC patients (Yamazaki, et al. (2014) Lab. Invest., 94:683). Activin A signals through canonical SMAD3-dependent pathways and/or non-canonical pathways which involves p38 phosphorylation (Loomans, et al. (2014) Cancers 7:70). To understand the potential signaling pathway of activin A in PDAC, the phosphorylation status of tumor SMAD3 and p38 proteins were analyzed. Inhba siRNA significantly reduced SMAD3 phosphorylation but p38 phosphorylation was statistically not-significant when compared to the Sc-si group (FIG. 5B), indicating that activin A might act through SMAD3 phosphorylation during orthotopic tumor growth. Next, IHC for pSMAD3, ACVR2A, and ACVR2B was performed to identify target cells of activin A in tumors. pSMAD3 signal was detected in the nuclei of most cells in the tumors from Sc-si. Furthermore, ACVR2A and ACVR2B staining showed a consistent pattern with pSMAD3, but the ACVR2A signal was weaker than ACVR2B. These findings indicate that activin A targets both cancer cells and CAFs in the present model. Additionally, high expression of pSMAD3 and ACVR2B in tumor-associated ADM regions was observed, indicating a role of activin A in tumor-mediated ADM.

In addition, the activin A source was determined through IHC staining with the inhibin βA antibody on the tumor section. Inhibin βA was intensely detected in cancer cells and fibroblasts. In addition, high expression of inhibin βA was detected in tumor-associated ADM region. Therefore, tumor and systemic activin A levels would originate from cancer cells, fibroblasts, and ADM as consistent with IHC findings in TMAs.

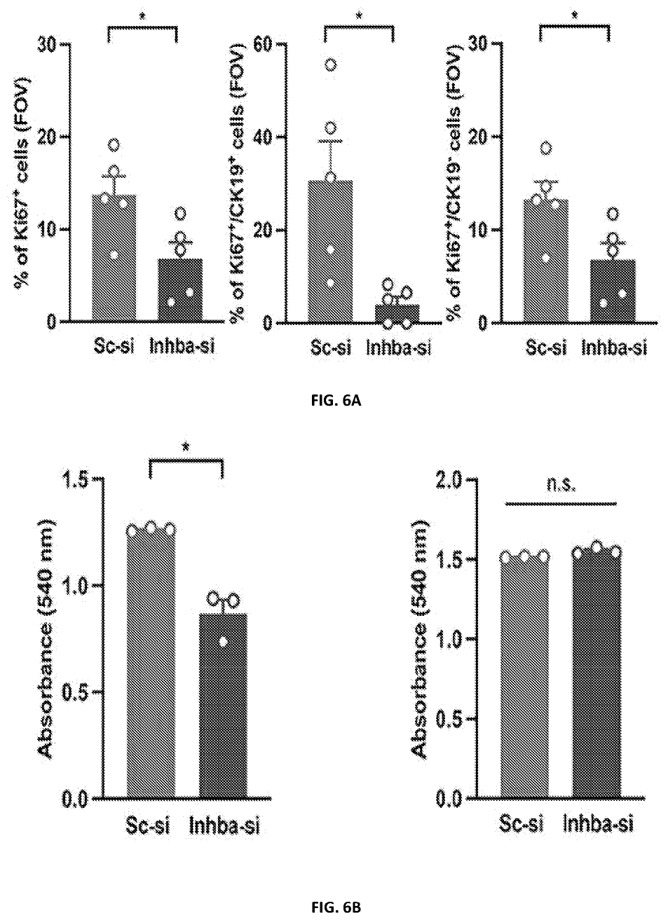

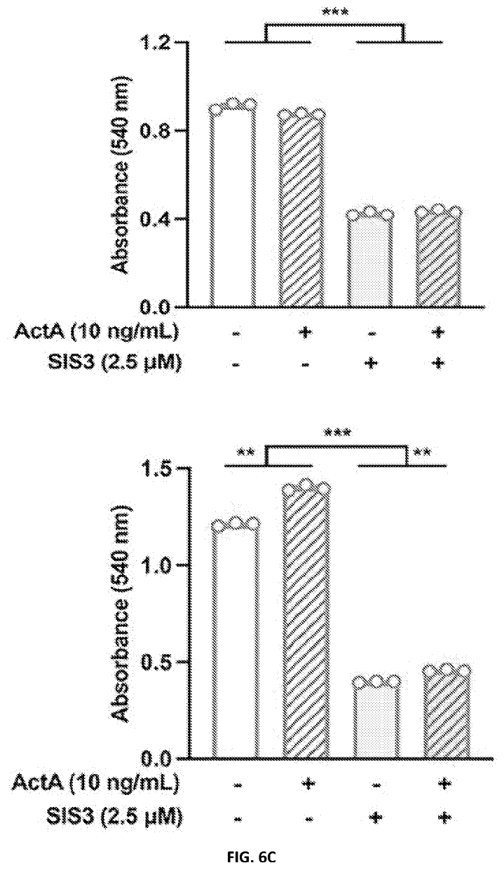

Next, it was investigated if inhba knockdown decreases cancer proliferation which would mediate stalled tumor growth in vivo. IF staining showed that Ki67-positive cells were abundantly detected in the tumors from the Sc-si group. Co-staining with CK19 revealed that Ki67 was detected in CK19-positive and CK19-negative cells in the tumors from the Sc-si group. However, Ki67 was barely detected in the tumors from the inhba-si group. The total number of Ki67-positive cells and the number of Ki67-positive cells among CK19-positive or negative cells was quantified (FIG. 6A). When compared to the Sc-si group, the inhba-si group had a significantly reduced total number of Ki67-positive cells and number of Ki67-positive cells among CK19-positive cells in the tumors (FIG. 6A). The number of Ki67-positive cells among CK19-negative cells was also significantly lower in the inhba-si group. These data indicate that activin A upregulates the proliferation of cancer and cancer-associated cells in PDAC tumors.

It was further investigated whether inhba siRNA directly suppresses the proliferation of PDAC tumor cells using KPC8069 and KPC1245 which showed the highest and lowest activin A secretion (FIG. 2A). When compared to the cells treated with scramble siRNA, inhba siRNA significantly decreased the proliferation of KPC8069 but showed no inhibitory effects on KPC1245 proliferation (FIG. 6B). Inversely, exogenous activin A had no effects on KPC8069 proliferation but significantly enhanced KPC1245 proliferation. SIS3, a selective inhibitor of SMAD3 phosphorylation, greatly decreased the proliferation of both KPC8069 and KPC1245 in the presence or absence of exogenous activin A (FIG. 6C). These findings indicate that inhba siRNA directly suppresses the proliferation of a subset of inhba-high KPC cells that reciprocally promote the proliferation of inhba-low tumor cells.

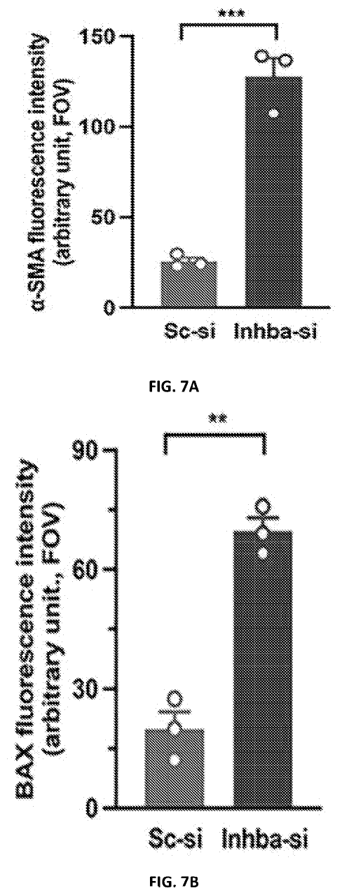

Because SMAD3 phosphorylation was observed in stromal cells, it was determined whether inhba knockdown influences CAF activation states and subsequent T cell infiltration which would be an alternative pathway to hinder tumor growth and metastasis. Interestingly, tumor fibroblasts from the Sc-si group had a weak expression of α-SMA. However, α-SMA was intensely and abundantly found in the cells especially surrounding CK19-positive cells in the tumors from the inhba-si group. Quantification of α-SMA fluorescence indicates considerably high expression of α-SMA in the inhba-si group (FIG. 7A). The knockout of Prrx1 promotes the accumulation of α-SMA^{high} fibroblasts (Feldmann, et al. (2021) Gastroenterology 160:346). PRRX1 was abundantly expressed in the nuclei of CK19-positive and CK19-negative cells in tumors from the Sc-si group, whereas PRRX1 was weakly expressed, and the majority of weak PRRX1 signaling was detected in CK19-positive cells in tumors from inhba-si group. In addition, increased infiltration of α-SMA^{high} fibroblasts correlated with high infiltration of T cells. Pan T cells (CD3-positive) and cytotoxic T cells (CD8-positive) were abundantly detected in the tumor beds from the inhba-si group, but those cell types were almost absent in the tumor beds from the Sc-si group. Furthermore, apoptotic BAX expression was examined in tumor cells. Greatly increased BAX expression in CK19-positive cells was detected in the inhba-si group when compared to in Sc-si group. The intensity of BAX in CK19-positive cells was statistically higher in the inhba-si group than the Sc-si group (FIG. 7B). However, PD-L1 expression in CK19 cells remained stable in the inhba-si group when compared to the Sc-si group. These observations indicate that activin A suppression promoted the accumulation of α-SMA^{high} fibroblasts and increased cytotoxic T-cell infiltration and cancer apoptosis.

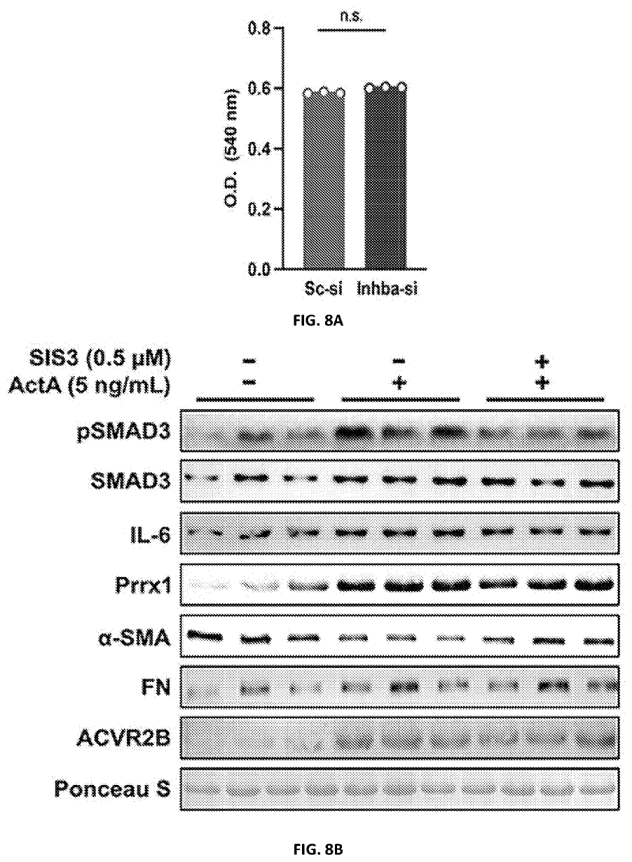

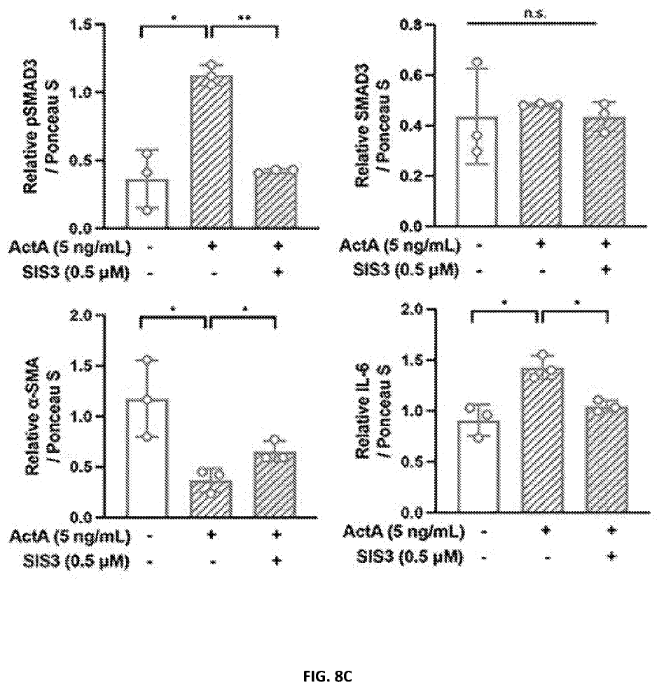

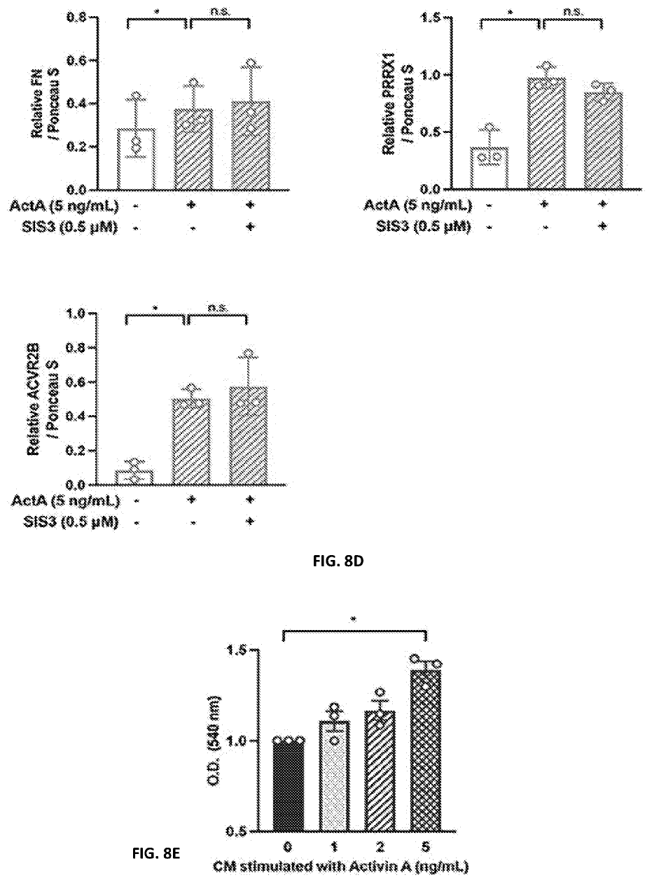

Because CAF activation status was skewed towards α-SMA^{high} fibroblasts, and fewer fibroblasts were accumulated in tumors by activin A suppression, the role of activin A in the proliferation and activation of PSC was examined. Although inhba siRNA did not affect the proliferation of primary mouse PSC (FIG. 8A), activin A significantly increased the expression levels of ACVR2B and SMAD3 phosphorylation and coincidently increased the expression levels of IL-6, PRRX1, and fibronectin (FN) but decreased α-SMA levels (FIGS. 8B, 8C). In the presence of SIS3, activin A failed to induce SMAD3 phosphorylation, upregulation of IL-6, and suppression of α-SMA. The expression of FN, PRRX1, and ACVR2B remained unchanged after being upregulated by activin A in the presence of SIS3 (FIGS. 8B, 8D). The upregulation of IL-6 and SMAD3 phosphorylation by activin A through the SMAD3 pathway was confirmed using IF staining.

It was also examined whether CM from PSC stimulated with activin A would promote KPC8069 proliferation. It was observed that the CM from PSC stimulated with activin A at 5 ng mL⁻¹ significantly promoted KPC8069 proliferation (FIG. 8E). Collectively, the in vitro observation indicates that activin A indirectly induces tumor proliferation through fibroblasts.

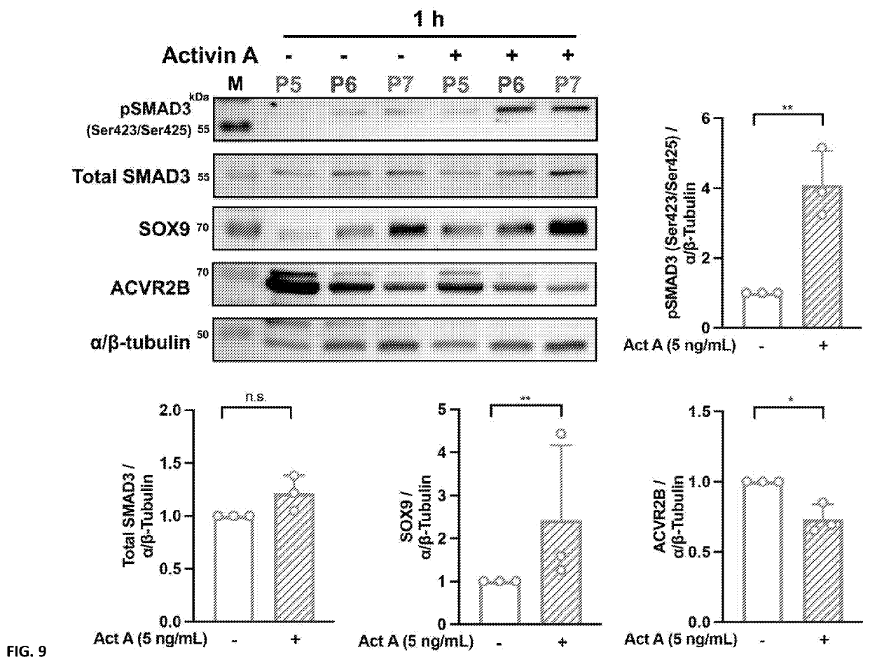

Abundant staining of pSMAD3 and ACVR2B in tumor-associated ADM indicated the role of activin A in tumor-associated ADM. As consistent with in vivo observations, activin A promoted phosphorylation of SMAD3 and expression of SOX9, a marker for ADM, in acinar 266-6 cells (FIG. 9). However, it is unclear if tumor-associated ADM becomes cancerous but will constitute PDAC tumor in part by secreting activin A and promoting the accumulation of fibroblasts as demonstrated herein and human invasive PDAC (Kibe, et al. (2019) Cancer Lett., 444:70).

Activin A is recognized as a prognostic factor for PDAC (Mancinelli, et al. (2021) Sci. Rep., 11:7986; Zhong, et al. (2019) J. Cachexia, Sarcopenia Muscle 10:1083). Here, it is demonstrated that gene expression of INHBA, not other subunit isoforms, was significantly upregulated in pancreatic tumors than in normal pancreatic tissues through secondary analysis of publicly accessible gene expression data. Regarding a source cell of activin A, the inhibin βA subunit is detected in acinar, stromal, and tumor cells of human pancreatic tumors in TMA (Mancinelli, et al. (2021) Sci. Rep., 11:7986). DAB staining with TMA indicates that the inhibin RA subunit was transiently overexpressed from atrophic acinar cells in chronic pancreatitis to the cells in PanIN, and was highly overexpressed in human PDAC. Consistent observations were made in KPC tumors and the orthotopic mice (Zhao, et al. (2020) Cancer Res., 80:3359). Moreover, tissue activin A levels were nearly 7 times higher in tumors than in other tissues. Thus, tumor and stromal cells are source cells of local and systemic activin A in PDAC.

As shown herein, activin A is a therapeutic target for PDAC beyond its recognition as a prognostic factor for PDAC. Herein, it is first reported that activin A suppression, which is acquired through tumor-targeted inhba siRNA delivery, retarded orthotopic tumor growth/metastasis and improved weight loss and survival. Without being bound by theory, in vitro data indicate that activin A promotes KPC cell proliferation through SMAD3 phosphorylation. Consistently, activin A promoted MIA-PaCa2 cell proliferation, however, INHBA overexpression showed no effects on heterotopic tumor growth in immunodeficient mice (Togashi, et al. (2015) Cancer Lett., 356:819). The null effects on tumor size by INHBA overexpression would be related to the model used, including subcutaneous cell implantation, lack of metastasis, and T-cell deficiency, all of which would potentially modulate tumor growth (Togashi, et al. (2015) Cancer Lett., 356:819; Saluja, et al. (2013) Gastroenterology 144:1194). Furthermore, the inhibitory effects of soluble ACVR2B/Fc on orthotopic tumor growth were not statistically significant, although a 30% reduction in tumor weight was observed (Zhong, et al. (2019) J. Cachexia, Sarcopenia Muscle 10:1083). The borderline significance would pertain to the use of KPC cells with low inhba expression for generating an orthotopic model which elevates plasma activin A level close to 1.5 ng mL⁻¹ (Zhong, et al. (2019) J. Cachexia, Sarcopenia Muscle 10:1083). Contrarily, the present model achieved activin A level of above 5 ng mL⁻¹ which is within the range of activin A detected in PDAC patients (Xu, et al. (2022) Sci. Rep., 12:1659). Furthermore, it was observed that inhba siRNA did not have effects on KPC1245 proliferation which expresses a relatively low inhba gene, whereas inhba siRNA suppressed the proliferation of KPC8069 which showed a relatively high inhba expression.

It was found that activin A modulates the activation state of CAFs in PDAC. Microenvironmental heterogeneity is a risk factor for metastasis and survival in PDAC patients and is related to the activation status of CAFs (Wang, et al. (2020) Am. J. Cancer Res., 10:1937; Truong, et al. (2021) Cancers 13:5028; Liu, et al. (2017) Cancer Cell Int., 17: 68). MyoCAFs show relatively higher expression levels of myofibroblast genes such as Acta2 for α-SMA. iCAFs express relatively higher levels of inflammatory genes such as Il-6. Both types are shown to be interconvertible (Ohlund, et al. (2017) J. Exp. Med., 214:579). In PDAC patients, a low histopathological score for α-SMA predicted poorer survival in PDAC patients (Ozdemir, et al. (2014) Cancer Cell 25:719). In PDAC mice, the deletion of Acta2-expressing CAFs significantly decreased survival (Ozdemir, et al. (2014) Cancer Cell 25:719). Conversely, overexpression of fibroblast α-SMA by Prrx1 knockout prevented PDAC metastasis in orthotopic PDAC mice (Feldmann, et al. (2021) Gastroenterology 160:346). Furthermore, the tumor beds enriched with α-SMA^{high} CAFs resulted in high infiltration of immune cells such as CD3-positive or CD8-positive T cells (Feldmann, et al. (2021) Gastroenterology 160:346). Therefore, a body of evidence highlights the importance of CAF activation status in association with tumor growth and metastasis. However, little is known regarding the potential regulators of CAF activation in PDAC. In the present model, tumor activin A suppression retarded tumor growth and metastasis. Activin A suppression increased the accumulation of α-SMA^{high}/PRRX1^{low} CAFs and led to the infiltration of CD3- or CD8-positive T cells with a concomitant increase of BAX expression in PDAC tumor cells. In in vitro data, it was confirmed that activin A increases the protein expression of IL-6 and PRRX1 but decreases α-SMA protein expression in PSCs. SMAD3 activation is a responsible target pathway for regulating IL-6 and α-SMA expression by activin A. Activin A-mediated PRRX1 upregulation seems to be mildly inhibited by SMAD3 inhibitor.

Activin A has tumor-suppressing roles in PDAC. The ablation of the Acvr1b gene promoted Kras^{G12D}-mediated tumorigenesis and decreased PDAC survival in mice (Qiu, et al. (2016) Gastroenterology 150: 218). Additionally, consistent observations were made by the loss of the Smad4 gene, in mice harboring Kras^{G12D} (Bardeesy, et al. (2006) Genes Dev., 20:3130). However, loss of Acvr1b or Smad4 occurs before Kras^{G12D}-dependent tumor development (Qiu, et al. (2016) Gastroenterology 150: 218; Bardeesy, et al. (2006) Genes Dev., 20:3130). Therefore, the evidence addresses the importance of activin A signaling in tumorigenesis, especially the development of PDAC precursor lesions. Accordingly, without being bound by theory, the loss of activin A signaling accelerates the occurrence rates of premalignant intraductal papillary mucinous neoplasia (IPMN), whereas Kras^{G12D} only elicits PanIN, another precursor lesion. Consistently, the treatment of soluble ACVR2B/Fc at 1.5 months old age, before PDAC develops, promoted the development of cystic lesions, which are reminiscent of a high incidence of IPMN by loss of activin A signaling during Kras^{G12D}-dependent tumorigenesis (Zhao, et al. (2020) Cancer Res., 80:3359; Qiu, et al. (2016) Gastroenterology 150: 218; Bardeesy, et al. (2006) Genes Dev., 20:3130; Guerra, et al. (2013) Mol. Oncol., 7:232). Therefore, the consistent observations identify activin A as a risk factor predisposing to the development of IPMN when KrasG12D is present.

It is demonstrated herein that activin A suppression renders favorable outcomes for PDAC including the mitigation of tumor growth/metastasis and weight wasting and improvement of survival in mice. Activin A promotes tumor cell proliferation and suppresses α-SMA expression in fibroblasts which coincidently promotes PDAC growth/metastasis and inhibits T cell infiltration. Without being bound by theory, in vitro data indicate that activin A plays its tumor-promoting roles in a SMAD3-dependent manner.

Collectively, the data indicate that inhba suppression impedes ADM mediated by KPC cells in mice and indicates activin A as one of the tumor-initiating factors. Thus, targeting and inhibiting INHBA is therapeutic for pancreatic cancer and in early PDAC development.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: An Inhibin βA (inhba) inhibitory nucleic acid molecule.

2: The inhba inhibitory nucleic acid molecule of claim 1, wherein said inhibitory nucleic acid molecule is an antisense oligonucleotide, siRNA, or shRNA.

3: The inhba inhibitory nucleic acid molecule of claim 1, wherein said inhibitory nucleic acid molecule targets a sequence in SEQ ID NO: 2 or SEQ ID NO: 3.

4: The inhba inhibitory nucleic acid molecule of claim 1, wherein said inhibitory nucleic acid molecule targets SEQ ID NO: 4, SEQ ID NO: 6, SEQ ID NO: 8, or SEQ ID NO: 10.

5: The inhba inhibitory nucleic acid molecule of claim 1, wherein said inhibitory nucleic acid molecule comprises a modification.

6: The inhba inhibitory nucleic acid molecule of claim 5, wherein said modification comprises a modified sugar group, a modified base, and/or modified phosphate group.

7: The inhba inhibitory nucleic acid molecule of claim 1, wherein said inhibitory nucleic acid molecule comprises at least one modification selected from the group consisting of modified nucleotide bases, locked nucleic acids (LNA), unlocked nucleic acids (UNA), 2″-deoxy, 2′-O-methyl, 2′-fluoro, 2′-methoxyethyl, 2′-aminoethyl, hypoxanthine, 2,4-di-fluorotoluene, dihydrouridine, 2′-thiouridine, pseudouridine, phosphorothioate, boranophosphate, and peptide nucleic acids (PNAs).

8: The inhba inhibitory nucleic acid molecule of claim 1, wherein said inhibitory nucleic acid molecule is contained in a vehicle.

9: The inhba inhibitory nucleic acid molecule of claim 8, wherein said vehicle is a nanoparticle, micelle, microparticle, lipid polymer, or hydrogel.

10: A nanoparticle comprising the inhba inhibitory nucleic acid molecule of claim 1.

11: A composition comprising an inhba inhibitory nucleic acid molecule of claim 1 and at least one pharmaceutically acceptable carrier.

12: A method of treating a disease or disorder in a subject in need thereof, said method comprising administering an inhba inhibitory nucleic acid molecule to the subject.

13: The method of claim 12, wherein said inhba inhibitory nucleic acid molecule is an inhba inhibitory nucleic acid molecule of claim 1.

14: The method of claim 12, wherein said disease or disorder is selected from the group consisting of cancers, autoimmune diseases, fibrotic disorders, blood disorders, allergies and allergic diseases, heart failure, neurodegenerative diseases, cachexia and inflammatory diseases.

15: The method of claim 12, wherein said disease or disorder is cancer.

16: The method of claim 14, wherein said cancer is pancreatic cancer.

17: The method of claim 14, wherein said cancer is pancreatic ductal adenocarcinoma.

18: The method of claim 12, wherein said disease or disorder is cachexia.

19: The method of claim 12, wherein said disease or disorder is fibrotic disease.

20: The method of claim 12, wherein treating said disease or disorder reduces or slows, and/or prevents a disease or disorder in a subject.

21: The method of claim 12, wherein said administering an inhba inhibitory nucleic acid molecule is by injection, oral, pulmonary, topical, or nasal administration.


SEQUENCE LISTING (53 sequences)

SEQ ID NO: 1 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 2 - DNA, 6046 bp, Homo sapiens
  source 1..6046: mol_type = other DNA
acagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaaaacaaacaacattaaaaaattgagaacaagtatggaaagaatgaaagatcaaggaaaaaagaataccaagttacatttcgttaaggtgcttatgatcttagaactatgcaacctaataggtttgaaactgtttacctgagagagaacaaaaagagagacttttttgtattggaagtaatctgattaatttttattttcttcaaggagagatacttgaaaggaatatgtttgtccatctgttggatccaaacatttctatattttgtaaatgttgttgttgttttttttttaatcgtttactatttgcactacaatggtgtttgacctgtctaatccttatttaacaagtattttctttggttgggggtgggggtggggtttaagagctgcacttaatgtgagctataaaagaactgctacagcacacaaaatagctatttttattattataattataattattattattattttgtaccttaaaaaatagacacatacaccaaagacatttgtgtgagcctttaaacagtctgtctgtggttggtatcattcaccatcaatgagtcaggggttgggattcaaggttgagtagtgtggattgtgttcaggcttaaaagacctgagaagtttggtttttgactccttttacatccatgaaacaggacatttcatactggatgtacagtagttgtacactgttggatatcaagttcaatcaaattcatggaactacatgcttgtatgtgtatatatacattgcttgtgcatatgcatatctgtatgtatatatacatgtattgtaccatgtccatacacattttaagcacttcaggctgtcattttttaatgttcttaaagcaatgaatgtttgtgtgcaaaacacagtatttttaagaaggataggctatagtttttgcttttactctgaactaggtgggcgcatttcaaaaattcggatgggaaaaagcctggaaattccagtgaatattcagcaaggccctctttcattgtacagggatcaaatttcctcctcttttttgtgccccctcccacttctacaagttatcccctgtggggaaaacaggatgataatcaaaactctgggctgatgtttttccaacttagtgtctattggaatcaatcttaaatcagaagctttttcagaaaaataatatttaggccagaattagagttgagtgtattttttaaaaatgattaaggcttggttgtgagaaatattacctgtaccagctgggaaaaataatgtcatcactaactaaaagataattaatttgagagaaagtgttaagagagggagagtaaggaagagaacagttaagaggaggcagaggtgagggcagtagtaaaaatctctaaaattttaatttacagccaaaattcttcatgtgtaaatttgtattgattcagatgcagaaatgaaaaaaaaacacctttgttttataaatatcaaagtacatgcttaaagccaagtttttatctagtttattctagtacttagcttgcctggaatagctaataaattattcatgtatgtgcttttgaaaatccagagccctatttttacacacttgtgtgaagttggcaaacattttgaaaaatggaaaaaagtttctaataattgggaacaattacattaattaatattttgtaaaatattgaagcttttagccctatgtcaatttgtagattaaaataaattaattataggaaaggaagataacagtgagaaaccaaacattacaaaaggtggtttagctctccttgaaaaatatactaagttggtatactataacacttggctatatgtaggcaatgtcactactgggcaaatacacttactgtgttctagaggcagccctttcttatgcagaaaatacaatacgcactgcatgagaagcttgagagtggattctaatccaggtctgtcgaccttggatatcatgcatgtgggaaggtgggtgtggtgagaaaagttttaaggcaagagtagatggccatgttcaactttacaaaatttcttggaaaactggcagtattttgaactgcatcttctttggtaccggaacctgcagaaacagtgtgagaaattaagtcctggttcactgcgcagtagcaaagatggtcaaggccatggaaaaagcagaaatttaccaagaaagctgatacccatgtatagttcccactcatctcaaatacatctgctatctttttaagctaagtcctagacatatcggggataacatgggggttgattagtgaccacagttatcagaagcagagaaatgtaattccatattttatttgaaacttattccatattttaattggatattgagtgattgggttatcaaacacccacaaactttaattttgttaaatttatatggctttgaaatagaagtataagttgctaccattttttgataacattgaaagatagtattttaccatctttaatcatcttggaaaatacaagtcctgtgaacaaccactctttcacctagcagcatgaggccaaaagtaaaggctttaaattataacatatgggattcttagtagtatgtttttttcttgaaactcagtggctctatctaaccttactatctcctcactctttctctaagactaaactctaggctcttaaaaatctgcccacaccaatcttagaagctctgaaaagaatttgtctttaaatatcttttaatagtaacatgtattttatggaccaaattgacattttcgactattttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaaggtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgta

SEQ ID NO: 3 - DNA, 1281 bp, Homo sapiens
  source 1..1281: mol_type = other DNA
atgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatag

SEQ ID NO: 4 - DNA, 25 bp, synthetic construct
  source 1..25: mol_type = other DNA
gtcaagaagcacattttaaacatgc

SEQ ID NO: 5 - DNA, 27 bp, synthetic construct
  source 1..27: mol_type = other DNA
gcatgtttaaaatgtgcttcttgacgg

SEQ ID NO: 6 - DNA, 25 bp, synthetic construct
  source 1..25: mol_type = other DNA
aacgcgatcagaaagcttcatgtgg

SEQ ID NO: 7 - DNA, 27 bp, synthetic construct
  source 1..27: mol_type = other DNA
ccacatgaagctttctgatcgcgttca

SEQ ID NO: 8 - DNA, 25 bp, synthetic construct
  source 1..25: mol_type = other DNA
ctaaaagtccccaaggccaacagga

SEQ ID NO: 9 - DNA, 27 bp, synthetic construct
  source 1..27: mol_type = other DNA
tcctgttggccttggggacttttagga

SEQ ID NO: 10 - DNA, 25 bp, synthetic construct
  source 1..25: mol_type = other DNA
atgatgatggtcaaaacatcatcaa

SEQ ID NO: 11 - DNA, 27 bp, synthetic construct
  source 1..27: mol_type = other DNA
ttgatgatgttttgaccatcatcatag

SEQ ID NO: 12 - DNA, 25 bp, synthetic construct
  source 1..25: mol_type = other DNA
gtcaagaagcacatcttaaacatgc

SEQ ID NO: 13 - DNA, 27 bp, synthetic construct
  source 1..27: mol_type = other DNA
gcatgtttaagatgtgcttcttgacag

SEQ ID NO: 14 - DNA, 25 bp, synthetic construct
  source 1..25: mol_type = other DNA
aacgcgatcagaaagcttcatgtgg

SEQ ID NO: 15 - DNA, 27 bp, synthetic construct
  source 1..27: mol_type = other DNA
ccacatgaagctttctgatcgcgttga

SEQ ID NO: 16 - DNA, 25 bp, synthetic construct
  source 1..25: mol_type = other DNA
ctgaaagtccccaaggctaacagaa

SEQ ID NO: 17 - DNA, 27 bp, synthetic construct
  source 1..27: mol_type = other DNA
ttctgttagccttggggactttcagga

SEQ ID NO: 18 - DNA, 25 bp, synthetic construct
  source 1..25: mol_type = other DNA
acgatgatggtcaaaacatcatcaa

SEQ ID NO: 19 - DNA, 27 bp, synthetic construct
  source 1..27: mol_type = other DNA
ttgatgatgttttgaccatcatcgtaa

SEQ ID NO: 20 - RNA, 25 nt, synthetic construct
  source 1..25: mol_type = other RNA
gtcaagaagcacattttaaacatgc

SEQ ID NO: 21 - RNA, 27 nt, synthetic construct
  source 1..27: mol_type = other RNA
gcatgtttaaaatgtgcttcttgacgg

SEQ ID NO: 22 - RNA, 25 nt, synthetic construct
  source 1..25: mol_type = other RNA
aacgcgatcagaaagcttcatgtgg

SEQ ID NO: 23 - RNA, 27 nt, synthetic construct
  source 1..27: mol_type = other RNA
ccacatgaagctttctgatcgcgttca

SEQ ID NO: 24 - RNA, 25 nt, synthetic construct
  source 1..25: mol_type = other RNA
ctaaaagtccccaaggccaacagga

SEQ ID NO: 25 - RNA, 27 nt, synthetic construct
  source 1..27: mol_type = other RNA
tcctgttggccttggggacttttagga

SEQ ID NO: 26 - RNA, 25 nt, synthetic construct
  source 1..25: mol_type = other RNA
atgatgatggtcaaaacatcatcaa

SEQ ID NO: 27 - RNA, 27 nt, synthetic construct
  source 1..27: mol_type = other RNA
ttgatgatgttttgaccatcatcatag

SEQ ID NO: 28 - RNA, 25 nt, synthetic construct
  source 1..25: mol_type = other RNA
gtcaagaagcacatcttaaacatgc

SEQ ID NO: 29 - RNA, 27 nt, synthetic construct
  source 1..27: mol_type = other RNA
gcatgtttaagatgtgcttcttgacag

SEQ ID NO: 30 - RNA, 25 nt, synthetic construct
  source 1..25: mol_type = other RNA
aacgcgatcagaaagcttcatgtgg

SEQ ID NO: 31 - RNA, 27 nt, synthetic construct
  source 1..27: mol_type = other RNA
ccacatgaagctttctgatcgcgttga

SEQ ID NO: 32 - RNA, 25 nt, synthetic construct
  source 1..25: mol_type = other RNA
ctgaaagtccccaaggctaacagaa

SEQ ID NO: 33 - RNA, 27 nt, synthetic construct
  source 1..27: mol_type = other RNA
ttctgttagccttggggactttcagga

SEQ ID NO: 34 - RNA, 25 nt, synthetic construct
  source 1..25: mol_type = other RNA
acgatgatggtcaaaacatcatcaa

SEQ ID NO: 35 - RNA, 27 nt, synthetic construct
  source 1..27: mol_type = other RNA
ttgatgatgttttgaccatcatcgtaa

SEQ ID NO: 36 - DNA, 20 bp, synthetic construct
  source 1..20: mol_type = other DNA
gcatcatcaacagtgcaacg

SEQ ID NO: 37 - DNA, 19 bp, synthetic construct
  source 1..19: mol_type = other DNA
gcagaaacactcgcagtca

SEQ ID NO: 38 - DNA, 24 bp, synthetic construct
  source 1..24: mol_type = other DNA
cctaccatcctatgagtgaatacc

SEQ ID NO: 39 - DNA, 21 bp, synthetic construct
  source 1..21: mol_type = other DNA
gactgctactgccattacctg

SEQ ID NO: 40 - DNA, 22 bp, synthetic construct
  source 1..22: mol_type = other DNA
cagcctctccaagaaatgtagt

SEQ ID NO: 41 - DNA, 22 bp, synthetic construct
  source 1..22: mol_type = other DNA
gacaagttagtcctgacatccc

SEQ ID NO: 42 - DNA, 22 bp, synthetic construct
  source 1..22: mol_type = other DNA
ccactttctccttctccacacc

SEQ ID NO: 43 - DNA, 22 bp, synthetic construct
  source 1..22: mol_type = other DNA
ggttgtcgatgcagccttgctt

SEQ ID NO: 44 - DNA, 22 bp, synthetic construct
  source 1..22: mol_type = other DNA
ccattgaagggctcatcaccac

SEQ ID NO: 45 - DNA, 21 bp, synthetic construct
  source 1..21: mol_type = other DNA
ccgttcttgtaccaggaaagg

SEQ ID NO: 46 - DNA, 21 bp, synthetic construct
  source 1..21: mol_type = other DNA
ttcttcccccttgttgtcctc

SEQ ID NO: 47 - DNA, 22 bp, synthetic construct
  source 1..22: mol_type = other DNA
acaggtgtagttggtctgtagg

SEQ ID NO: 48 - DNA, 22 bp, synthetic construct
  source 1..22: mol_type = other DNA
agacggtgatgctgagacacga

SEQ ID NO: 49 - DNA, 22 bp, synthetic construct
  source 1..22: mol_type = other DNA
gaccattccagccacagtcact

SEQ ID NO: 50 - DNA, 21 bp, synthetic construct
  source 1..21: mol_type = other DNA
tcatacctgcatgaggatgtg

SEQ ID NO: 51 - DNA, 18 bp, synthetic construct
  source 1..18: mol_type = other DNA
ccaagccaaagtcagcca

SEQ ID NO: 52 - DNA, 19 bp, synthetic construct
  source 1..19: mol_type = other DNA
cctggctgttcggtttgag

SEQ ID NO: 53 - DNA, 21 bp, synthetic construct
  source 1..21: mol_type = other DNA
gcgtctctctggaagttgatg